(19) United States
(12) Patent Application Publication
(10) Pub. No.: US 2003/0199528 A1
(43) Pub. Date: Oct. 23, 2003

(54) Hetrocyclic compounds that are inhibitors of the enzyme DPP-IV

(76) Inventors: Anders B. Kanstrup, Espergaerde (DK); Christian Klarner Sams, Frederiksberg C (DK); Jane Marie Lundbeck, Glostrup (DK); Lise Brown Christiansen, Lyngby (DK); Andrew Neil Bowler, Gentofte (DK)

Correspondence Address: Reza Green, Esq., Novo Nordisk Pharmaceuticals, Inc., 100 College Road West, Princeton, NJ, 08540, US

(21) Appl. No.: 10/353,181

(22) Filed: Jan. 28, 2003

Related U.S. Application Data
(63) Continuation of application No. PCT/DK02/00608, filed on Sep. 19, 2002.
(60) Provisional application No. 60/324,574, filed on Sep. 24, 2001.

(30) Foreign Application Priority Data
Sep. 19, 2001 (DK) PA 2001/01358

(51) Int. Cl. A61K 31/522; C07D 473/04

(52) U.S. Cl. 514/263.2; 544/276; 544/269; 514/263.35

(57) ABSTRACT

The present invention relates to therapeutically active and selective inhibitors of the enzyme DPP-IV, pharmaceutical compositions comprising the compounds and the use of such compounds for and the manufacture of medicaments for treating diseases that are associated with proteins that are subject to inactivation by DPP-IV, such as type 2 diabetes and obesity. The present inhibitors are novel purine derivatives, attached at position 8 of the purine skeleton to a diamine.

### CROSS-REFERENCE TO RELATED APPLICATIONS

This application is a continuation of application serial no. PCT/DK02/00608 filed on Sep. 19, 2002 and claims priority under 35 U.S.C. 119 of Danish application no. PA 2001 01358 filed on Sep. 24, 2001, and U.S. provisional application No. 60/324,574 filed on Sep. 24, 2001, the contents of all of which are fully incorporated herein by reference.

### FIELD OF THE INVENTION

The present invention relates to therapeutically active and selective inhibitors of the enzyme DPP-IV, pharmaceutical compositions comprising the compounds and the use of such compounds for and the manufacture of medicaments for treating diseases that are associated with proteins that are subject to inactivation by DPP-IV, such as type 2 diabetes and obesity.

### BACKGROUND OF THE INVENTION

Dipeptidyl peptidase-IV (DPP-IV), a serine protease belonging to the group of post-proline/alanine cleaving amino-dipeptidases, specifically removes the two N-terminal amino acids from proteins having proline or alanine in position 2.

Although the physiological role of DPP-IV has not been completely established, it is believed to play an important role in neuropeptide metabolism, T-cell activation, gastric ulceration, functional dyspepsia, obesity, appetite regulation, impaired fasting glucose (IFG) and diabetes.

DPP-IV has been implicated in the control of glucose metabolism because its substrates include the insulinotropic hormones Glucagon like peptide-1 (GLP-1) and Gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance. Therefore, such inhibitors have been proposed for the treatment of patients with Type 2 diabetes, a disease characterised by decreased glucose tolerance. (Hoist, J. J.; Deacon, C. F. Diabetes 47 (1998) 1663-70) Diabetic dyslipidemia is characterized by multiple lipoprotein defects, including moderately high serum levels of cholesterol and triglycerides, small LDL particles, and low levels of HDL cholesterol. The results of recent clinical trials reveal beneficial effects of cholesterol-lowering therapy in diabetic and non-diabetic patients, thus supporting increased emphasis on treatment of diabetic dyslipidemia. The National Cholesterol Education Program's Adult Treatment Panel II advocated this need for intensive treatment of diabetic dyslipidemia. Obesity is a well-known risk factor for the development of many very common diseases such as atherosclerosis, hypertension and diabetes. The incidence of obese people and thereby also these diseases is increasing throughout the entire industrialised world. Except for exercise, diet and food restriction no convincing pharmacological treatment for reducing body weight effectively and acceptably currently exist. However, due to its indirect but important effect as a risk factor in mortal and common diseases it will be important to find treatment for obesity or appetite regulation. Even mild obesity increases the risk for premature death, diabetes, hypertension, atherosclerosis, gallbladder disease and certain types of cancer. In the industrialised western world the prevalence of obesity has increased significantly in the past few decades. Because of the high prevalence of obesity and its health consequences, its prevention and treatment should be a high public health priority.

At present a variety of techniques are available to effect initial weight loss. Unfortunately, initial weight loss is not an optimal therapeutic goal. Rather, the problem is that most obese patients eventually regain their weight. An effective means to establish and/or sustain weight loss is the major challenge in the treatment of obesity today.

Several compounds have been shown to inhibit DPP-IV, but all of these have limitations in relation to the potency, stability, selectivity, toxicity, and pharmacodynamic properties. Thus, there is a need for the provision of DPP-IV inhibitors that are superior with respect to one or more of the above listed properties, and which will be useful for the treatment of conditions, which may be regulated or normalised by inhibition of DPP-IV.

### SUMMARY OF THE INVENTION

The present invention consists of novel purine derivatives, attached at position 8 of the purine skeleton to a diamine. The compounds of the present invention are thus not amino acid derivatives, such as the presently known DPP-IV inhibitors, but consist of structural elements hitherto unrelated to DPP-IV inhibition, and as such they represent novel solutions to the problem of finding an optimal DPP-IV inhibitor. These compounds are potent and selective inhibitors of DPP-IV, and are effective in treating conditions that may be regulated or normalised via inhibition of DPP-IV. The invention also concerns methods for preparing the compounds, pharmaceutical compositions comprising the compounds, a method of inhibiting DPP-IV comprising administering to a patient in need of such treatment a therapeutically effective amount thereof, the compounds for use as a pharmaceutical, and their use in a process for the preparation of a medicament for treating a condition which may be regulated or normalised via inhibition of DPP-IV.

Definitions

The term “DPP-IV” as used herein is intended to mean Dipeptidyl peptidase IV (EC 3.4.14.5; DPP-IV), also known as CD26. DPP-IV cleaves a dipeptide from the N terminus of a polypeptide chain containing a proline or alanine residue in the penultimate position.

The term “treatment” is defined as the management and care of a patient for the purpose of combating the disease, condition, or disorder and includes the administration of a compound of the present invention to prevent the onset of the symptoms or complications, or alleviating the symptoms or complications, or eliminating the disease, condition, or disorder.

The term “beta cell degeneration” is intended to mean loss of beta cell function, beta cell dysfunction, and death of beta cells, such as necrosis or apoptosis of beta cells.

The term “alkyl” as used herein, alone or in combination, refers to a straight or branched, saturated hydrocarbon chain having the indicated number of carbon atoms. Similarly the term “alkylene” refers to the corresponding bivalent radical having the indicated number of carbon atoms.

Non-limiting examples of such saturated hydrocarbons are e.g. methyl, ethyl, n-propyl, iso-propyl, n-butyl, sec. Butyl, isobutyl, tert. Butyl, n-pentyl, 2-methylbutyl, 3-methylbutyl, n-hexyl, 4-methylpentyl, neopentyl, 2,2-dimethylpropyl and the like.

The term “alkenyl” used herein, alone or in combination, refers to a straight or branched, unsaturated hydrocarbon chain having the indicated number of carbon atoms and at least one double bond. Similarly the term “alkenylene” refers to the corresponding bivalent radical having the indicated number of carbon atoms. Non-limiting examples of such unsaturated hydrocarbons are vinyl, 1-propenyl, allyl, isopropenyl, n-butenyl, n-pentenyl and n-hexenyl and the like.

The term “alkynyl” as used herein, alone or in combination, refers to an unsaturated hydrocarbon chain having the indicated number of carbon atoms and at least one triple bond such as but not limited to —C≡CH, —C≡CCH₃, —CH₂C≡CH, —CH₂—CH₂—C≡CH, —CH(CH₃)C≡CH and the like.

The term “cycloalkyl” as used herein refers to a radical of one or more saturated cyclic hydrocarbon having the indicated number of carbon atoms. Similarly the term “cycloalkylene” refers to the corresponding bivalent radical having the indicated number of carbon atoms. Non-limiting examples of such saturated cyclic hydrocarbons are cyclopropyl, cyclobutyl, cyclopentyl, cyclohexyl, adamantyl and the like.

The term “cycloheteroalkyl” as used herein refers to a radical of totally saturated heterocycle having the indicated number of carbon atoms like a cyclic hydrocarbon containing one or more heteroatoms selected from nitrogen, oxygen and sulphur independently in the cycle. Similarly the term “cycloheteroalkylene” refers to the corresponding bivalent radical having the indicated number of carbon atoms like a cyclic hydrocarbon containing one or more heteroatoms selected from nitrogen, oxygen and sulphur independently in the cycle.

Non-limiting examples of such saturated heterocycles are pyrrolidine (1-pyrrolidine; 2-pyrrolidine; 3-pyrrolidine; 4-pyrrolidine; 5-pyrrolidine); pyrazolidine (1-pyrazolidine; 2-pyrazolidine; 3-pyrazolidine; 4-pyrazolidine; 5-pyrazolidine); imidazolidine (1-imidazolidine; 2-imidazolidine; 3-imidazolidine; 4-imidazolidine; 5-imidazolidine); thiazolidine (2-thiazolidine; 3-thiazolidine; 4-thiazolidine; 5-thiazolidine); piperidine (1-piperidine; 2-piperidine; 3-piperidine; 4-piperidine; 5-piperidine; 6-piperidine); piperazine (1-piperazine; 2-piperazine; 3-piperazine; 4-piperazine; 5-piperazine; 6-piperazine); morpholine (2-morpholine; 3-morpholine; 4-morpholine; 5-morpholine; 6-morpholine); thiomorpholine (2-thiomorpholine; 3-thiomorpholine; 4-thiomorpholine; 5-thiomorpholine; 6-thiomorpholine); 1,2-oxathiolane (3-(1,2-oxathiolane); 4-(1,2-oxathiolane); 5-(1,2-oxathiolane); 1,3-dioxolane (2-(1,3-dioxolane); 4-(1,3-dioxolane); 5-(1,3-dioxolane); tetrahydropyrane; (2-tetrahydropyrane; 3-tetrahydropyrane; 4-tetrahydropyrane; 5-tetrahydropyrane; 6-tetrahydropyrane); hexahydropyridazine (1-(hexahydropyridazine); 2-(hexahydropyridazine); 3-(hexahydropyridazine); 4-(hexahydropyridazine); 5-(hexahydropyridazine); 6-(hexahydropyridazine)). Similarly the term “cycloheteroalkylene” refers to the corresponding bivalent radical having the indicated number of carbon atoms like a cyclic hydrocarbon containing one or more heteroatoms selected from nitrogen, oxygen and sulphur independently in the cycle.

The term “aryl” as used herein includes carbocyclic aromatic ring systems. Aryl is also intended to include the partially hydrogenated derivatives of the carbocyclic systems. Similarly the term “arylene” refers to the corresponding bivalent radical.

The term “heteroaryl” as used herein includes heterocyclic unsaturated ring systems containing one or more heteroatoms selected from nitrogen, oxygen and sulphur. Similarly the term “heteroarylenearylene” refers to the corresponding bivalent radical.

Non-limiting examples of such unsaturated ring systems containing one or more heteroatoms are furyl, thienyl, pyrrolyl. The term “heteroaryl” is also intended to include the partially hydrogenated derivatives of the heterocyclic systems enumerated below.

The terms “aryl” and “heteroaryl” as used herein refers to an aryl which can be optionally substituted or a heteroaryl which can be optionally substituted and includes phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydrobenzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydrobenzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The term “halogen” as used herein refers to fluoro, chloro, bromo, and iodo.

The term “arylene-alkylene” as used herein refers to an “arylene” group as defined above attached through an “alkylene” group as defined above having the indicated number of carbon atoms. Similarly the term “alkylene-arylene” as used herein refers to an “alkylene” group as defined above having the indicated number of carbon atoms attached through an “arylene” group as defined above.

The term “alkylene-arylene-alkylene” refers to a “arylene-alkylene” group as defined above connected through an “alkylene” group as defined above having the indicated number of carbon atoms.

The term “heteroaryl-alkylene” as used herein refers to a “heteroaryl” group as defined above attached through an “alkylene” group as defined above having the indicated number of carbon atoms.

The term “cycloalkyl-alkylene” as used herein refers to a “cycloalkyl” group as defined above having the indicated number of carbon atoms attached through an “alkylene” group as defined above having the indicated number of carbon atoms.

The term “cycloheteroalkyl-alkylene” as used herein refers to a “cycloheteroalkyl” group as defined above having the indicated number of carbon atoms attached through an “alkylene” group as defined above having the indicated number of carbon atoms.

### DESCRIPTION OF THE INVENTION

The present invention provides compounds of formula I

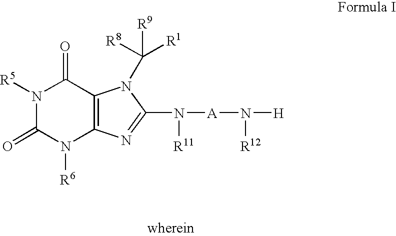

A is C₂-C₆ alkylene; C₂-C₁₀ alkenylene; C₃-C₇ cycloalkylene; C₃-C₇ cycloheteroalkylene; arylene; heteroarylene; C₁-C₂ alkylene-arylene; arylene-C₁-C₂ alkylene; C₁-C₂ alkylene-arylene-C₁-C₂ alkylene, wherein each alkylene, alkenylene, cycloalkylene, cycloheteroalkylene, arylene, or heteroarylene is optionally substituted with one or more R³ independently;

R¹ is aryl optionally substituted with one or more R² independently or heteroaryl optionally substituted with one or more R² independently;

R² is H; C₁-C₇ alkyl; C₂-C₇ alkenyl; C₂-C₇ alkynyl; C₃-C₇ cycloalkyl; C₃-C₇ cycloheteroalkyl; —NHCOR³; —NHSO₂R³; —SR³; —SOR³; —SO₂R³; —OCOR³; —CO₂R⁴; —CON(R⁴)₂; —CSN(R⁴)₂; —NHCON(R⁴)₂; —NHCSN(R⁴)₂; —NHCONNH₂; —SO₂N(R⁴)₂; —OR⁴; cyano; nitro; halogen, wherein each alkyl, alkenyl, alkynyl, cycloalkyl and cycloheteroalkyl is optionally substituted with one or more R³ independently;

R³ is C₁-C₁₀ alkyl; C₂-C₁₀ alkenyl; C₂-C₁₀ alkynyl; C₃-C₇ cycloalkyl; aryl; heteroaryl; OR¹⁰; N(R¹⁰)₂; SR¹⁰, wherein each alkyl, alkenyl, alkynyl, cycloalkyl, aryl and heteroaryl is optionally substituted with one or more R¹⁰ independently;

R⁴ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; aryl-C₁-C₅ alkylene; heteroaryl; heteroaryl-C₁-C₅alkylene, wherein each alkyl, alkenyl, alkynyl, cycloalkyl, cycloheteroalkyl, aryl, aryl-C₁-C₅ alkylene, heteroaryl, and heteroaryl-C₁-C₅ alkylene is optionally substituted with one or more R¹⁰ independently;

R⁵ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; —OR⁷; —[(CH₂)ₒ—O]ₚ—C₁-C₅alkyl, wherein o and p are 1-3 independently, and wherein each alkyl, alkenyl, alkynyl, cycloalkyl, cycloheteroalkyl, aryl, and heteroaryl is optionally substituted with one or more R⁷ independently;

R⁶ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; aryl-C₁-C₅ alkylene; heteroaryl-C₁-C₅ alkylene; C₃-C₇ cycloheteroalkyl-C₁-C₅ alkylene, wherein each alkyl, alkenyl, alkynyl, cycloalkyl, cycloheteroalkyl, C₃-C₇ cycloheteroalkyl-C₁-C₅ alkylene, aryl, aryl-C₁-C₅ alkylene, heteroaryl, aryl-C₁-C₅ alkylene, and heteroaryl-C₁-C₅ alkylene is optionally substituted with one or more R¹⁰ independently;

R⁷ is H; ═O; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; SR¹⁰; cyano; hydroxy; halogen; —CF₃; —CCl₃; —OCF₃; or —OCH₃ wherein each alkyl, alkenyl, alkynyl, cycloalkyl, cycloheteroalkyl, aryl, and heteroaryl is optionally substituted with one or more R¹⁰ independently;

R⁸ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; SR¹⁰, wherein each alkyl, alkenyl, alkynyl, cycloalkyl, cycloheteroalkyl, aryl, and heteroaryl is optionally substituted with one or more R¹⁰ independently;

R⁹ is H; C₁-C₁₀ alkyl optionally substituted with one or more R⁸ independently; or halogen;

R¹⁰ is H; —CF₃; —CCl₃; —OCF₃; —OCH₃; cyano; halogen; —OH, —COCH₃; —CONH₂; —CONHCH₃; —CON(CH₃)₂; —NO₂; —SO₂NH₂; or —SO₂N(CH₃)₂;

if two R⁴ or two R¹⁰ are attached to the same nitrogen they may be connected to form a 3- to 7-membered ring;

R¹¹ is H; C₁-C₆ alkyl optionally substituted with one or more R³ independently;

R¹² is H; C₁-C₆ alkyl optionally substituted with one or more R³ independently; or

If A is C₃-C₇ cycloalkylene or C₃-C₇ cycloheteroalkylene R¹² may be a valence bond between the nitrogen to which R¹² is attached and one of the atoms in the cycloalkylene or cycloheteroalkylene;

or a salt thereof with a pharmaceutically acceptable acid or base.

In another embodiment A is C₂-C₆ alkylene; C₂-C₁₀ alkenylene; C₃-C₇ cycloalkylene; C₃-C₇ cycloheteroalkylene; or arylene, wherein each alkylene, alkenylene, cycloalkylene, cycloheteroalkylene, or arylene is optionally substituted with one or more R³ independently;

In another embodiment A is C₃-C₇ cycloalkylene optionally substituted with one or more R³ independently.

In another embodiment A is cyclohexylene optionally substituted with one or more R³ independently.

In another embodiment A is cyclohexylene.

In another embodiment R¹ is aryl optionally substituted with one or more R² independently.

In another embodiment R¹ is phenyl optionally substituted with one or more R² independently.

In another embodiment R₂ is C₁-C₇ alkyl; C₂-C₇ alkynyl; cyano; or halogen, wherein each alkyl and alkynyl is optionally substituted with one or more R³ independently.

In another embodiment R₂ is C₁-C₇ alkyl; C₂-C₇ alkynyl; cyano; or halogen.

In another embodiment R₂ is halogen.

In another embodiment R³ is C₁-C₁₀ alkyl or aryl, wherein each alkyl or aryl is substituted with one or more R¹⁰ independently.

In another embodiment R³ is C₁-C₁₀ alkyl or aryl.

In another embodiment R³ is methyl or phenyl.

In another embodiment R⁴ is H; C₁-C₁₀ alkyl or aryl, wherein each alkyl or aryl is substituted with one or more R¹⁰ independently.

In another embodiment R⁴ is H; C₁-C₁₀ alkyl or aryl.

In another embodiment R⁴ is H, methyl or phenyl.

In another embodiment R⁵ is H; C₁-C₁₀ alkyl; aryl-C₁-C₅ alkylene; or heteroaryl-C₁-C₅ alkylene, wherein each alkyl, aryl-C₁-C₅ alkylene and heteroaryl-C₁-C₅ alkylene is optionally substituted with one or more R⁷ independently.

In another embodiment R⁵ is H; C₁-C₁₀ alkyl optionally substituted with one or more R⁷ independently; or C₂-C₁₀ alkenyl optionally substituted with one or more R⁷ independently.

In another embodiment R⁵ is H or C₁-C₁₀ alkyl optionally substituted with one or more R⁷ independently.

In another embodiment R⁵ is H.

In another embodiment R⁵ is methyl or ethyl optionally substituted with one or more R⁷ independently.

In another embodiment R⁶ is C₁-C₁₀ alkyl; aryl-C₁-C₅ alkylene; or heteroaryl-C₁-C₅ alkylene, wherein each alkyl, aryl-C₁-C₅ alkylene and heteroaryl-C₁-C₅ alkylene is optionally substituted with one or more R¹⁰ independently.

In another embodiment R⁶ is C₁-C₁₀ alkyl; aryl-C₁-C₅ alkylene; or heteroaryl-C₁-C₅ alkylene.

In another embodiment R⁶ is C₁-C₁₀ alkyl optionally substituted with one or more R¹⁰ independently.

In another embodiment R⁶ is C₁-C₁₀ alkyl.

In another embodiment R⁶ is methyl or ethyl optionally substituted by one or more R¹⁰ independently.

In another embodiment R⁷ is H; ═O; C₁-C₁₀ alkyl; C₃-C₇ cycloalkyl; C₃-C₇ cycloheteroalkyl; aryl; heteroaryl, OR¹⁰; N(R¹⁰)₂; SR¹⁰, wherein each alkyl, cycloalkyl, cycloheteroalkyl, aryl, and heteroaryl is optionally substituted with one or more R¹⁰ independently.

In another embodiment R⁷ is ═O; C₃-C₇ cycloalkyl; C₃-C₇ cycloheteroalkyl; aryl; or heteroaryl, wherein each cycloalkyl, cycloheteroalkyl, aryl, and heteroaryl is optionally substituted with one or more R¹⁰ independently.

In another embodiment R⁷ is ═O; C₃-C₇ cycloalkyl optionally substituted with one or more R¹⁰ independently or aryl optionally substituted with one or more R¹⁰ independently.

In another embodiment R⁷ is ═O or aryl optionally substituted with one or more R¹⁰ independently.

In another embodiment R⁷ is ═O or phenyl optionally substituted by one or more R¹⁰ independently.

In another embodiment R⁸ is aryl or heteroaryl, wherein each aryl and heteroaryl is optionally substituted with one or more R¹⁰ independently.

In another embodiment R⁸ is aryl or heteroaryl.

In another embodiment R⁸ is phenyl.

In another embodiment R⁹ is H; C₁-C₁₀ alkyl; or halogen.

In another embodiment R⁹ is H.

In another embodiment R¹⁰ is H; —CF₃; —OH; cyano; halogen; —OCF₃; or —OCH₃.

In another embodiment R¹⁰ is H; cyano; halogen; or —OCH₃.

In another embodiment R¹¹ is H.

In another embodiment R¹² is H.

In another embodiment the invention provides compounds of the general formula II

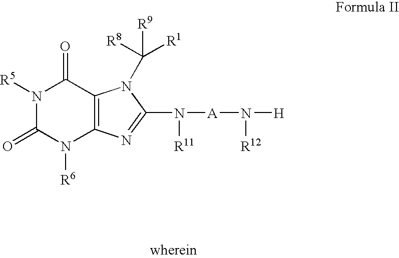

A is C₂-C₆ alkylene; C₂-C₁₀ alkenylene; C₃-C₇ cycloalkylene; C₃-C₇ cycloheteroalkylene; arylene; heteroarylene; C₁-C₂ alkylene-arylene; arylene-C₁-C₂ alkylene; C₁-C₂ alkylene-arylene-C₁-C₂ alkylene, wherein each alkylene, alkenylene, cycloalkylene, cycloheteroalkylene, arylene, or heteroarylene is optionally substituted with one or more R³ independently;

R¹ is aryl optionally substituted with one or more R² independently or heteroaryl optionally substituted with one or more R² independently;

R² is H; C₁-C₇ alkyl; C₂-C₇ alkenyl; C₂-C₇ alkynyl; C₃-C₇ cycloalkyl; C₃-C₇ cycloheteroalkyl; —NHCOR³; —NHSO₂R³; —SR³; —SOR³; —SO₂R³; —OCOR³; —CO₂R⁴; —CON(R⁴)₂; —CSN(R⁴)₂; —NHCON(R⁴)₂; —NHCSN(R⁴)₂; —NHCONNH₂; —SO₂N(R⁴)₂; —OR⁴; cyano; —CF₃; nitro; halogen, wherein each alkyl, alkenyl, alkynyl, cycloalkyl and cycloheteroalkyl is optionally substituted with one or more R³ independently;

R³ is C₁-C₁₀ alkyl; C₂-C₁₁ alkenyl; C₂-C₁₁ alkynyl; C₃-C₇ cycloalkyl; aryl; heteroaryl; OR¹⁰; N(R¹⁰)₂; SR¹⁰, wherein each alkyl, alkenyl, alkynyl, cycloalkyl, aryl and heteroaryl is optionally substituted with one or more R¹⁰ independently;

R⁴ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; aryl-C₁-C₅ alkylene; heteroaryl; heteroaryl-C₁-C₅alkylene, —CF₃ or —CHF₂, wherein each alkyl, alkenyl, alkynyl, cycloalkyl, cycloheteroalkyl, aryl, aryl-C₁-C₅ alkylene, heteroaryl, and heteroaryl-C₁-C₅ alkylene is optionally substituted with one or more R¹⁰ independently;

R⁵ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; —OR⁷; aryl-C₁-C₅alkylene; heteroaryl-C₁-C₅alkylene; —C₁-C₅-alkyl-C(═O)-aryl, —C₁-C₅-alkyl-C(═O)-heteroaryl or —[(CH₂)ₒ—O]ₚ—C₁-C₅ alkyl; wherein o and p are 1-3 independently, and wherein each alkyl, alkenyl, alkynyl, cycloalkyl, cycloheteroalkyl, aryl, heteroaryl, aryl-C₁-C₅ alkylene; —C₁-C₅-alkyl-C(═O)-aryl, —C₁-C₅-alkyl-C(═O)-heteroaryl and heteroaryl-C₁-C₅ alkylene is optionally substituted with one or more R⁷ independently;

R⁶ is H; C₁-C₁₀ alkyl; C₂-C₁₀alkenyl; C₂-C₁₀alkynyl; C₃-C₇ cycloalkyl; C₃-C₇ cycloheteroalkyl; aryl; heteroaryl; aryl-C₁-C₅ alkylene; heteroaryl-C₁-C₅ alkylene; C₃-C₇ cycloheteroalkyl-C₁-C₅ alkylene, wherein each alkyl, alkenyl, alkynyl, cycloalkyl, cycloheteroalkyl, C₃-C₇ cycloheteroalkyl-C₁-C₅ alkylene, aryl, heteroaryl, aryl-C₁-C₅ alkylene, and heteroaryl-C₁-C₅ alkylene is optionally substituted with one or more R¹⁰ independently;

R⁷ is H; ═O; C₁-C₁₀ alkyl; C₂-C₁₀alkenyl; C₂-C₁₀alkynyl; C₃-C₇ cycloalkyl; C₃-C₇ cycloheteroalkyl; aryl; heteroaryl, OR¹⁰; N(R¹⁰)₂; SR¹⁰; cyano; hydroxy; halogen; —CF₃; —CCl₃; —OCF₃; or —OCH₃ wherein each alkyl, alkenyl, alkynyl, cycloalkyl, cycloheteroalkyl, aryl, and heteroaryl is optionally substituted with one or more R¹⁰ independently;

R⁸ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; SR¹⁰, wherein each alkyl, alkenyl, alkynyl, cycloalkyl, cycloheteroalkyl, aryl, and heteroaryl is optionally substituted with one or more R¹⁰ independently;

R⁹ is H; C₁-C₁₀ alkyl optionally substituted with one or more R⁸ independently; or halogen;

R¹⁰ is H; —CF₃; —CCl₃; —OCF₃; —OCH₃; cyano; halogen; —OH, —COCH₃; —CONH₂; —CONHCH₃; —CON(CH₃)₂; —NO₂; —SO₂NH₂; or —SO₂N(CH₃)₂;

if two R⁴ or two R¹⁰ are attached to the same nitrogen they may be connected to form a 3- to 7-membered ring;

R¹¹ is H; C₁-C₆ alkyl optionally substituted with one or more R³ independently;

R¹² is H; C₁-C₆ alkyl optionally substituted with one or more R³ independently; or

If A is C₃-C₇ cycloalkylene or C₃-C₇ cycloheteroalkylene R¹² may be a valence bond between the nitrogen to which R¹² is attached and one of the atoms in the cycloalkylene or cycloheteroalkylene;

or a salt thereof with a pharmaceutically acceptable acid or base.

In another embodiment A is C₂-C₆ alkylene; C₂-C₁₀ alkenylene; C₃-C₇ cycloalkylene; C₃-C₇ cycloheteroalkylene; or arylene, wherein each alkylene, alkenylene, cycloalkylene, cycloheteroalkylene, or arylene is optionally substituted with one or more R³ independently;

In another embodiment A is C₂-C₆ alkylene; C₂-C₁₀ alkenylene; C₃-C₇ cycloalkylene; C₃-C₇ cycloheteroalkylene; arylene; heteroarylene; C₁-C₂ alkylene-arylene; arylene-C₁-C₂ alkylene; C₁-C₂ alkylene-arylene-C₁-C₂ alkylene, wherein each alkylene, alkenylene, cycloalkylene, cycloheteroalkylene, arylene, or heteroarylene is optionally substituted with one or more R³ independently;

R¹ is aryl optionally substituted with one or more R² independently or heteroaryl optionally substituted with one or more R² independently;

R² is H; C₁-C₇ alkyl; C₂-C₇ alkenyl; C₂-C₇ alkynyl; C₃-C₇ cycloalkyl; C₃-C₇ cycloheteroalkyl; —NHCOR³; —NHSO₂R³; —SR³; —SOR³; —SO₂R³; —OCOR³; —CO₂R⁴; —CON(R⁴)₂; —CSN(R⁴)₂; —NHCON(R⁴)₂; —NHCSN(R⁴)₂; —NHCONNH₂; —SO₂N(R⁴)₂; —OR⁴; cyano; nitro; halogen, wherein each alkyl, alkenyl, alkynyl, cycloalkyl and cycloheteroalkyl is optionally substituted with one or more R³ independently;

R³ is C₁-C₁₀ alkyl; C₂-C₁₀ alkenyl; C₂-C₁₀ alkynyl; C₃-C₇ cycloalkyl; aryl; heteroaryl; OR¹⁰; N(R¹⁰)₂; SR¹⁰, wherein each alkyl, alkenyl, alkynyl, cycloalkyl, aryl and heteroaryl is optionally substituted with one or more R¹⁰ independently;

R⁴ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; aryl-C₁-C₅ alkylene; heteroaryl; heteroaryl-C₁-C₅ alkylene, wherein each alkyl, alkenyl, alkynyl, cycloalkyl, cycloheteroalkyl, aryl, aryl-C₁-C₅ alkylene, heteroaryl, and heteroaryl-C₁-C₅ alkylene is optionally substituted with one or more R¹⁰ independently;

R⁵ is H; C₁-C₁₀alkyl; C₂-C₁₀alkenyl; C₂-C₁₀ alkynyl; C₃-C₇ cycloalkyl; C₃-C₇ cycloheteroalkyl; aryl; heteroaryl; —OR⁷; —[(CH₂)ₒ—O]ₚ—C₁-C₅ alkyl, wherein o and p are 1-3 independently, and wherein each alkyl, alkenyl, alkynyl, cycloalkyl, cycloheteroalkyl, aryl, and heteroaryl is optionally substituted with one or more R⁷ independently;

R⁶ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; aryl-C₁-C₅ alkylene; heteroaryl-C₁-C₅ alkylene; C₃-C₇ cycloheteroalkyl-C₁-C₅ alkylene, wherein each alkyl, alkenyl, alkynyl, cycloalkyl, cycloheteroalkyl, C₃-C₇ cycloheteroalkyl-C₁-C₅ alkylene, aryl, aryl-C₁-C₅ alkylene, heteroaryl, aryl-C₁-C₅ alkylene, and heteroaryl-C₁-C₅ alkylene is optionally substituted with one or more R¹⁰ independently;

R⁷ is H; ═O; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; SR¹⁰; cyano; hydroxy; halogen; —CF₃; —CCl₃; —OCF₃; or —OCH₃ wherein each alkyl, alkenyl, alkynyl, cycloalkyl, cycloheteroalkyl, aryl, and heteroaryl is optionally substituted with one or more R¹⁰ independently;

R⁸ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; SR¹⁰, wherein each alkyl, alkenyl, alkynyl, cycloalkyl, cycloheteroalkyl, aryl, and heteroaryl is optionally substituted with one or more R¹⁰ independently;

R⁹ is H; C₁-C₁₀ alkyl optionally substituted with one or more R⁸ independently; or halogen;

R¹⁰ is H; —CF₃; —CCl₃; —OCF₃; —OCH₃; cyano; halogen; —OH, —COCH₃; —CONH₂; —CONHCH₃; —CON(CH₃)₂; —NO₂; —SO₂NH₂; or —SO₂N(CH₃)₂;

if two R⁴ or two R¹⁰ are attached to the same nitrogen they may be connected to form a 3- to 7-membered ring;

R¹¹ is H; C₁-C₆ alkyl optionally substituted with one or more R³ independently;

R¹² is H; C₁-C₆ alkyl optionally substituted with one or more R³ independently; or

If A is C₃-C₇ cycloalkylene or C₃-C₇ cycloheteroalkylene R¹² may be a valence bond between the nitrogen to which R¹² is attached and one of the atoms in the cycloalkylene or cycloheteroalkylene;

or a salt thereof with a pharmaceutically acceptable acid or base

In another embodiment A is C₃-C₇ cycloalkylene optionally substituted with one or more R³ independently.

In another embodiment A is cyclohexylene or cycloheptylene, each optionally substituted with one or more R³ independently.

In another embodiment A is cyclohexylene optionally substituted with one or more R³ independently

In another embodiment A is cyclohexylene or cycloheptylene.

In another embodiment A is cyclohexylene

In another embodiment A is

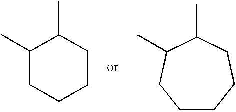

In another embodiment R¹ is aryl optionally substituted with one or more R² independently.

In another embodiment R¹ is phenyl optionally substituted with one or more R² independently.

In another embodiment R₂ is C₁-C₇ alkyl; C₂-C₇ alkynyl;; —OR⁴; cyano; —CF₃; or halogen, wherein each alkyl and alkynyl is optionally substituted with one or more R³ independently.

In another embodiment R₂ is C₁-C₇ alkyl; C₂-C₇ alkynyl; cyano; —CF₃; or halogen.

In another embodiment R₂ is cyano, —CF₃ or halogen.

In another embodiment R₂ is C₁-C₇ alkyl; C₂-C₇ alkynyl; cyano; or halogen, wherein each alkyl and alkynyl is optionally substituted with one or more R³ independently.

In another embodiment R₂ is C₁-C₇ alkyl; C₂-C₇ alkynyl; cyano; or halogen.

In another embodiment R₂ is halogen.

In another embodiment R³ is C₁-C₁₀ alkyl or aryl, wherein each alkyl or aryl is substituted with one or more R¹⁰ independently.

In another embodiment R³ is C₁-C₁₀ alkyl or aryl.

In another embodiment R³ is methyl or phenyl.

In another embodiment R⁴ is H; C₁-C₁₀ alkyl, —CHF₂, or aryl, wherein each alkyl or aryl is substituted with one or more R¹⁰ independently.

In another embodiment R⁴ is H; C₁-C₁₀ alkyl, —CHF₂, or aryl.

In another embodiment R⁴ is H, —CHF₂, methyl or phenyl.

In another embodiment R⁴ is H; CG-C₁-C₁₀ alkyl or aryl, wherein each alkyl or aryl is substituted with one or more R¹⁰ independently.

In another embodiment R⁴ is H; C₁-C₁₀ alkyl or aryl.

In another embodiment R⁴ is H, methyl or phenyl.

In another embodiment R⁵ is H; C₁-C₁₀ alkyl; aryl-C₁-C₅ alkylene; —C₁-C₅-alkyl-C(═O)-aryl; or heteroaryl-C₁-C₅ alkylene, wherein each alkyl, aryl-C₁-C₅ alkylene and heteroaryl-C₁-C₅alkylene is optionally substituted with one or more R⁷ independently.

In another embodiment R⁵ is H; C₁-C₁₀ alkyl optionally substituted with one or more R⁷ independently; —C₁-C₅-alkyl-C(═O)-aryl optionally substituted with one or more R⁷ independently or C₂-C₁₀ alkenyl optionally substituted with one or more R⁷ independently.

In another embodiment R⁵ is H, —C₁-C₅-alkyl-C(═O)-aryl optionally substituted with one or more R⁷ independently or C₁-C₁₀ alkyl optionally substituted with one or more R⁷ independently.

In another embodiment R⁵ is H or —C₁-C₅-alkyl-C(═O)-phenyl optionally substituted with one or more R⁷ independently.

In another embodiment R⁵ is methyl or ethyl optionally substituted with one or more R⁷ independently.

In another embodiment R⁵ is H; C₁-C₁₀ alkyl; aryl-C₁-C₅ alkylene; or heteroaryl-C₁-C₅ alkylene, wherein each alkyl, aryl-C₁-C₅ alkylene and heteroaryl-C₁-C₅ alkylene is optionally substituted with one or more R⁷ independently.

In another embodiment R⁵ is H; C₁-C₁₀ alkyl optionally substituted with one or more R⁷ independently; or C₂-C₁₀ alkenyl optionally substituted with one or more R⁷ independently.

In another embodiment R⁵ is H or C₁-C₁₀ alkyl optionally substituted with one or more R⁷ independently.

In another embodiment R⁵ is H

In another embodiment R⁵ is methyl

In another embodiment R⁵ is C₁-C₁₀ alkyl; aryl-C₁-C₅ alkylene; or heteroaryl-C₁-C₅ alkylene, wherein each alkyl, aryl-C₁-C₅ alkylene and heteroaryl-C₁-C₅ alkylene is optionally substituted with one or more R¹⁰ independently.

In another embodiment R⁶ is C₁-C₁₀ alkyl; aryl-C₁-C₅ alkylene; or heteroaryl-C₁-C₅ alkylene.

In another embodiment R⁶ is C₁-C₁₀ alkyl optionally substituted with one or more R¹⁰ independently.

In another embodiment R⁵ is C₁-C₁₀ alkyl.

In another embodiment R⁶ is methyl or ethyl optionally substituted by one or more R¹⁰ independently.

In another embodiment R⁶ is methyl

In another embodiment R⁷ is H; ═O; C₁-C₁₀ alkyl; C₃-C₇ cycloalkyl; C₃-C₇ cycloheteroalkyl; aryl; heteroaryl, OR¹⁰; N(R¹⁰)₂; SR¹⁰, cyano; or halogen, wherein each alkyl, cycloalkyl, cycloheteroalkyl, aryl, and heteroaryl is optionally substituted with one or more R¹⁰ independently.

In another embodiment R⁷ is ═O; OR¹⁰; C₃-C₇ cycloalkyl; C₃-C₇ cycloheteroalkyl; aryl; heteroaryl; cyano; or halogen, wherein each cycloalkyl, cycloheteroalkyl, aryl, and heteroaryl is optionally substituted with one or more R¹⁰ independently.

In another embodiment R⁷ is ═O; OR¹⁰; cyano; halogen; C₃-C₇ cycloalkyl optionally substituted with one or more R¹⁰ independently or aryl optionally substituted with one or more R¹⁰ independently.

In another embodiment R⁷ is H; ═O; C₁-C₁₀ alkyl; C₃-C₇ cycloalkyl; C₃-C₇ cycloheteroalkyl; aryl; heteroaryl, OR¹⁰; N(R¹⁰)₂; SR¹⁰, wherein each alkyl, cycloalkyl, cycloheteroalkyl, aryl, and heteroaryl is optionally substituted with one or more R¹⁰ independently.

In another embodiment R⁷ is ═O; C₃-C₇ cycloalkyl; C₃-C₇ cycloheteroalkyl; aryl; or heteroaryl, wherein each cycloalkyl, cycloheteroalkyl, aryl, and heteroaryl is optionally substituted with one or more R¹⁰ independently.

In another embodiment R⁷ is ═O; C₃-C₇ cycloalkyl optionally substituted with one or more R¹⁰ independently or aryl optionally substituted with one or more R¹⁰ independently

In another embodiment R⁷ is ═O or aryl optionally substituted with one or more R¹⁰ independently.

In another embodiment R⁷ is ═O or phenyl optionally substituted by one or more R¹⁰ independently.

In another embodiment R⁸ is aryl or heteroaryl, wherein each aryl and heteroaryl is optionally substituted with one or more R¹⁰ independently.

In another embodiment R⁸ is aryl or heteroaryl.

In another embodiment R⁸ is phenyl.

In another embodiment R⁹ is H; C₁-C₁₀ alkyl; or halogen.

In another embodiment R⁹ is H.

In another embodiment R¹⁰ is H; —CF₃; —OH; cyano; halogen; —OCF₃; or —OCH₃.

In another embodiment R¹⁰ is H; cyano; halogen; or —OCH₃.

In another embodiment R¹¹ is H.

In another embodiment R¹² is H.

Compounds of either formula I or formula 11 may be used for the manufacture of a medicament for treating diseases associated with proteins that are subject to inactivation by DPP-IV.

A further aspect of the invention is the use of a compound of the invention for the manufacture of a medicament for treating a condition that may be regulated or normalised via inhibition of DPP-IV.

Another aspect of the invention is the use of a compound of the invention for the manufacture of a medicament for treatment of metabolic disorders.

Another aspect of the invention is the use of a compound of the invention for the manufacture of a medicament for blood glucose lowering.

Another aspect of the invention is the use of a compound of the invention for the manufacture of a medicament for treatment of Type 2 diabetes

Another aspect of the invention is the use of a compound of the invention for the manufacture of a medicament for the treatment of impaired glucose tolerance (IGT).

Another aspect of the invention is the use of a compound of the invention for the manufacture of a medicament for the treatment of impaired fasting glucose (IFG).

Another aspect of the invention is the use of a compound of the invention for the manufacture of a medicament for prevention of hyperglycemia.

Another aspect of the invention is the use of a compound of the invention for the manufacture of a medicament for delaying the progression of impaired glucose tolerance (IGT) to Type 2 diabetes.

Another aspect of the invention is the use of a compound of the invention for the manufacture of a medicament for delaying the progression of non-insulin requiring Type 2 diabetes to insulin-requiring Type 2 diabetes.

Another aspect of the invention is the use of a compound of the invention for the manufacture of a medicament for increasing the number and/or the size of beta cells in a mammalian subject.

Another aspect of the invention is the use of a compound of the invention for the manufacture of a medicament for treatment of beta cell degeneration, in particular apoptosis of beta cells.

Another aspect of the invention is the use of a compound of the invention for the manufacture of a medicament for the treatment of disorders of food intake.

Another aspect of the invention is the use of a compound of the invention for the manufacture of a medicament for the treatment of obesity.

Another aspect of the invention is the use of a compound of the invention for the manufacture of a medicament for appetite regulation or induction of satiety.

Another aspect of the invention is the use of a compound of the invention for the manufacture of a medicament for the treatment of dyslipidemia.

Another aspect of the invention is the use of a compound of the invention for the manufacture of a medicament for treatment of functional dyspepsia, in particular irritable bowel syndrome.

A further aspect of the invention is a method for treating any one of the conditions mentioned above by administering to a subject in need thereof an effective amount of a compound of the invention.

A further aspect of the invention is a pharmaceutical composition suitable for treating any one of the conditions mentioned above comprising a compound of the invention.

The compounds of the present invention may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66, 2 (1977) that are known to the skilled artisan.

Also intended as pharmaceutically acceptable acid addition salts are the hydrates that the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

It is to be understood that the invention extends to all of the stereo isomeric forms of the claimed compounds, as well as the racemates.

Pharmaceutical Compositions

In another aspect, the present invention includes within its scope pharmaceutical compositions comprising, as an active ingredient, at least one compound of the invention which inhibits the enzymatic activity of DPP-IV or a pharmaceutically acceptable salt or prodrug or hydrate thereof together with a pharmaceutically acceptable carrier or diluent.

Pharmaceutical compositions containing a compound of the invention of the present invention may be prepared by conventional techniques, e.g. as described in Remington: The Science and Practice of Pharmacy, 19th Ed., 1995. The compositions may appear in conventional forms, for example capsules, tablets, aerosols, solutions, suspensions or topical applications.

Typical compositions include a compound of the invention which inhibits the enzymatic activity of DPP-IV or a pharmaceutically acceptable basic addition salt or prodrug or hydrate thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier which may be in the form of an ampoule, capsule, sachet, paper, or other container. When the carrier serves as a diluent, it may be solid, semi-solid, or liquid material which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, peanut oil, olive oil, gelatine, lactose, terra alba, sucrose, dextrin, magnesium carbonate, sugar, cyclodextrin, amylose, magnesium stearate, talc, gelatine, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include welting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compounds. The route of administration may be any route, which effectively transports the active compound of the invention which inhibits the enzymatic activity of DPP-IV to the appropriate or desired site of action, such as oral, nasal, pulmonary, buccal, subdermal, intradermal, transdermal or parenteral e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of the invention which inhibits the enzymatic activity of DPP-IV, dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil. Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet which may be prepared by conventional tabletting techniques may contain:

| Core: | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

The compounds of the invention are effective over a wide dosage range. For example, in the treatment of adult humans, dosages from about 0.05 to about 1000 mg, preferably from about 0.1 to about 500 mg, per day may be used. A most preferable dosage is about 0.5 mg to about 250 mg per day. In choosing a regimen for patients it may frequently be necessary to begin with a higher dosage and when the condition is under control to reduce the dosage. The exact dosage will depend upon the mode of administration, on the therapy desired, form in which administered, the subject to be treated and the body weight of the subject to be treated, and the preference and experience of the physician or veterinarian in charge.

Generally, the compounds of the present invention are dispensed in unit dosage form comprising from about 0.05 to about 1000 mg of active ingredient together with a pharmaceutically acceptable carrier per unit dosage.

Usually, dosage forms suitable for oral, nasal, pulmonal or transdermal administration comprise from about 0.05 mg to about 1000 mg, preferably from about 0.5 mg to about 250 mg of the compounds admixed with a pharmaceutically acceptable carrier or diluent.

The invention also encompasses prodrugs of a compound of the invention which on administration undergo chemical conversion by metabolic processes before becoming active pharmacological substances. In general, such prodrugs will be functional derivatives of a compound of the invention which are readily convertible in vivo into a compound of the invention. Conventional procedures for the selection and preparation of suitable prodrug derivatives are described, for example, in “Design of Prodrugs”, ed. H. Bundgaard, Elsevier, 1985.

Combination Treatments

The invention furthermore relates to the use of a compound according to the present invention for the preparation of a medicament for use in the treatment of diabetes in a regimen which additionally comprises treatment with another antidiabetic agent.

In the present context the expression “antidiabetic agent” includes compounds for the treatment and/or prophylaxis of insulin resistance and diseases wherein insulin resistance is the pathophysiological mechanism.

In one embodiment of this invention, the antidiabetic agent is insulin or GLP-1 or any analogue or derivative thereof.

In another embodiment the antidiabetic agent is a hypoglycemic agent, preferably an oral hypoglycemic agent.

Oral hypoglycemic agents are preferably selected from the group consisting of sulfonylureas, non-sulphonylurea insulin secretagogues, biguamides, thiazolidinediones, alpha glucosidase inhibitors, glucagon antagonists, GLP-1 agonists, potassium channel openers, insulin sensitizers, hepatic enzyme inhibitors, glucose uptake modulators, compounds modifying the lipid metabolism, compounds lowering food intake, and agents acting on the ATP-dependent potassium channel of the β-cells.

Among the sulfonylureas, tolbutamide, glibenclamide, glipizide and gliclazide are preferred.

Among the non-sulphonylurea insulin secretagogues, repaglinide and nateglinide are preferred.

Among the biguamides, mefformin is preferred.

Among the thiazolidinediones, troglitazone, rosiglitazone and ciglitazone are preferred.

Among the glucosidase inhibitors, acarbose is preferred.

Among the agents acting on the ATP-dependent potassium channel of the β-cells the following are preferred: glibenclamide, glipizide, gliclazide, repaglinide.

The cyclic amines used in the synthesis of the compounds of the invention are either commercially available, or have been made using published procedures. Racemic 3-aminopiperidine was made from 3-aminopyridine by reduction with PtO₂ (Nienburg. Chem. Ber. 70(1937)635). Enantiopure (R)— and (S)-3-aminopiperidine and (R)— and (S)-3-Aminopyrrolidine was made according to Moon, S—H and Lee, S. Synth. Commun. 28(1998)3919.

Pharmacological Methods

Methods for Measuring the Activity of Compounds which Inhibit the Enzymatic Activity of CD26/DPP-IV

### SUMMARY

Chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of α-cumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

Materials:

The following reagents and cells are commercially available:

Porcine CD26/DPP-IV (Sigma D-7052), Gly-Pro-pNA (Sigma G0513).

Assay buffer: 50 mM Tris pH 7.4, 150 mM NaCl, 0.1% Triton X-100.

Gly-Pro-pNA Cleavage-Assay for CD26:

The activity of purified CD26/DPP-IV is assayed in reactions containing:

70 μl assay buffer

10 μl inhibitor or buffer

10 μl substrate (Gly-Pro-pNA from a 0.1 M stock solution in water) or buffer

10 μl enzyme or buffer

Reactions containing identical amounts of enzyme, but varying concentrations of inhibitor and substrate, or buffer as control, are set up in parallel in individual wells of a 96-well ELISA plate. The plate is incubated at 25° C. and absorbance is read at 405 nm after 60 min incubation. The inhibitor constants are calculated by non-linear regression hyperbolic fit and the result is expressed as inhibition constant (Ki) in nM.

Diabetes Model

The Zucker Diabetic Fatty (ZDF) rat model can be used to investigate the effects of the compounds of the invention on both the treatment and prevention of diabetes as rats of this sub-strain are initially pre-diabetic although develop severe type 2 diabetes characterised by increased HbA1c levels over a period of 6 weeks. The same strain can be used to predict the clinical efficacy of other anti-diabetic drug types. For example, the model predicts the potency and limited clinical efficacy of thiazolidinedione insulin sensitizers compounds.

Chemical Methods

Preparative HPLC (Method A1)

Column: 1.9×15 cm Waters XTerra RP-18. Buffer: linear gradient 5-95% in 15 min, MeCN, 0.1% TFA, flow rate of 15 ml/min. The pooled fractions are either evaporated to dryness in vacuo, or evaporated in vacuo until the MeCN is removed, and then frozen and freeze dried.

Preparative HPLC (Method A2)

Column: Supelcosil ABZ+Plus, 25 cm×10 mm, 5 μm. Solvent A: 0.1% TFA/Water, solvent B: MeCN. Eluent composition: 5 min. 100% A, linear gradient 0-100% B in 7 min, 100% B in 2 min. Flow rate 5 ml/min. The column is allowed to equilibrate for 4 min in 100% A before the next run.

Preparative HPLC (Method A3)

The LC system consists of a Gilson 321 pump, 235 injector and 215-fraction collector equipped with a Waters Xterra 7.8 mm*100 mm column run with a gradient from 10% aqueous acetonitrile with 0.01% TFA to 100% acetonitrile with 0.01% TFA over 11 min. Flow rate 10 ml/min. The effluent is split 1:1000 to an Agilent 1100 MSD by a LC Packings ACM 10-50 flow splitter. The MS is equipped with an Agilent fraction collector kit, from which the analogue signal from extracted the target ion, is used for controlling fraction collection.

HPLC-MS (Method B1)

Column: Waters Xterra MS C-18×3 mm id. Buffer: Linear gradient 10-100% in 7.5 min, MeCN, 0.01% TFA, flow rate 1.0 ml/min. Detection 210 nm (analog output from diode array detector), MS-detection ionisation mode API-ES, scan 100-1000 amu step 0.1 amu.

HPLC-MS (Method B2)

Column: 0.3 mm×15 cm Waters Symmetry C₁₈. Buffer: Linear gradient 5-90% in 15 min, MeCN, 0.05% TFA, flow rate 1 ml/min

Analytical Separation of Stereoisomers (Method C)

CCE. Chiral capillary electrophoresis: Conditions: HP 3D Capillary Electrophoresis: 48.5/40 cm, 50 μm HP bubble capillary, Electrolyte: HS-p-CD (Regis) (2% w/v) in 50 mM phosphate buffer pH 2.5 (HP), Voltage: −17 kV, Injection: 30 mbar for 5 s.

Preparative Separation of Stereoisomers (Method D)

Analytical separations were performed on Hewlett Packard 1090 HPLC equipment with 5 chiral Daicel columns (AD, OD, AS, OJ and Welko-02, 250×4.6 mm) with a diode array detector. The mobile phases were 2-propanol:heptane mixtures with 0.1% DEA.

Preparative separations were performed with the above-mentioned type of columns (250×20 mm) on a preparative Gilson HPLC set-up. Relevant fractions were collected and evaporated (SpeedVac).

Microwave Assisted Reactions (Method F)

The reactants are mixed in an appropriate solvent in a closed Teflon vessel (XP 1500 Plus Vessel set) and heated in a micro wave oven (CEM MARSX microwave instrument. Magnetron frequency: 2455 MHz. Power Output: 1200 Waft.). The reaction mixture is cooled and evaporated in vacuo. Normally solvents like MeOH; EtOH, iPrOH; H₂O; DMF and DMSO are used.

Abbreviations

| | |
|---|---|
| DCHMA | Dicyclohexylmethylamine |
| DCM | Dichloromethane |
| DEA | Diethylamine |
| DIEA | Diisopropylethylamine |
| DMF | Dimethylformamide |
| DMSO | Dimethyl sulfoxide |
| EtOAc | Ethyl acetate |
| HOAc | Acetic acid |
| MeCN | Acetonitrile |
| TFA | Trifluoroacetic acid |
| THF | Tetrahydrofuran |
| TMG | Tetramethylguanidine |

Preparation of Cis-Cycloheptane-1,2-Diamine:

Step A: 2-Bromo-Cycloheptanone

Cycloheptanon (26 ml, 0.22 mmol) was dissolved in acetic acid (25 ml) and water (35 ml) and heated to 50° C. Bromine (11, 1 ml, 0.22 mmol) was added drop wise, and the reaction was cooled to room temperature. Potassium carbonate (50 g) was added in small portions, and the solution was poured into water (200 ml). The aqueous layer was extracted with dichloromethane (1×400 ml and 2×200 ml). The combined organics were washed with water (150 ml), dried over sodium sulphate, filtered and evaporated to afford 2-bromocycloheptanone.

Yield: 21.4 g, (50%).

¹H-NMR (CDCl₃, 200 MHz) δ=4.4(1H, q); 3,75 (1H, m), 2.5-1.3 (10H, m).

Step B: 3,4,5,6,7,8-Hexahydro-1H-cycloheptaimidazol-2-one

Urea (6,54 g, 108.86 mmol) and diethyleneglycol diethylether (10 ml) were heated to reflux and 2-bromo-cycloheptanone (10.4 g, 54.43 mmol) was added drop wise. The mixture was stirred 2 hours at 140° C., and then cooled to room temperature. Water (20 ml) was added and the precipitate was collected by filtration. The crystals were recrystallized from boiling ethanol to afford 3,4,5,6,7,8-hexahydro-1H-cycloheptaimidazol-2-one.

Yield: 1.64 g, (20%).

HPLC-MS (Method B1): m/z=153 (M+1); Rₜ=1.843 min.

Step C: Cis-Octahydro-cycloheptaimidazol-2-one 3,4,5,6,7,8-Hexahydro-1H-cycloheptaimidazol-2-one (1,62 g, 10.64 mmol) was suspended in ethanol (60 ml) and Raney Nickel was added under a nitrogen atmosphere. The mixture was stirred in a hydrogen atmosphere at 135° C. and 55 bar for 20 hours. The reaction mixture was filtered and washed with ethanol, and the filtrate was evaporated to afford cis-otahydrocycloheptaimidazol-2-one as crystals.

Yield: 1.3 g, (79%).

HPLC-MS (Method B1): m/z=155 (M+1); Rₜ=1.77 min.

Step D: Cis-Cycloheptane-1,2-diamine

cis-Otahydro-cycloheptaimidazol-2-one (1.30 g, 8.43 mmol) was dissolved in 65% sulphuric acid (15,8 ml) and heated to 145° C. for 2 days. The reaction mixture was cooled to room temperature and water (40 ml) was added. The mixture was added 50% sodium hydroxide until pH=10. The organic material was extracted into diethyl ether (4×350 ml), and the combined organic layers were dried with sodium sulphate, filtered and evaporated to afford the title compound.

Yield: 950 mg (88%).

HPLC-MS (Method B1): m/z=129 (M+1); Rₜ=0.53 min.

Preparation of 8-bromo-3-methyl-3,7-dihydropurine-2,6-dione

Step A: N-(6-Amino-1-methyl-2,4-dioxo-1,2,3,4-tetrahydropyrimidin-5-yl)formamide

Formic acid (400 ml) was cooled to 4° C. and 6-amino-1-methyluracil (50 g, 355 mol) was added. Sodium nitrite (24.42 g, 354 mol) was added in small portions over 10 minutes, and the mixture was stirred 3 hours at 10° C. The mixture was heated to 35° C. and platin on carbon (708 mg), water (18.7 ml), and formic acid (75 ml) were added. The reaction was stirred for 2 days and then filtered, and the solvents were evaporated. The crude product was crystallised from acetone to afford N-(6-amino-1-methyl-2,4-dioxo-1,2,3,4-tetrahydropyrimidin-5-yl)formamide.

Yield: 68.4 g (99%).

HPLC-MS (Method B2): m/z=185 (M+1); Rₜ=0.506 min.

Step B: 3-Methyl-3,7-dihydropurine-2,6-dione

N-(6-amino-1-methyl-2,4-dioxo-1,2,3,4-tetrahydropyrimidin-5-yl)formamide (68.4 g, 371 mol) and 2.5M sodium hydroxide (400 ml) were heated to 80° C. for 2 hours. The mixture was allowed to cool to room temperature and 6M hydrochloric acid (180 ml) was added (pH=2). The precipitate was collected by filtration to afford 3-methyl-3,7-dihydropurine-2,6-dione.

Yield: 36.7 g (60%).

HPLC-MS (Method B2): m/z=167 (M+1); Rₜ=0.571 min.

Step C: 8-Bromo-3-methyl-3,7-dihydro-purine-2,6-dione

3-Methyl-3,7-dihydropurine-2,6-dione (36.7 g, 221 mmol) and acetic acid (700 ml) were refluxed, and sodium acetate (39.1 g, 288 mmol) was added. The mixture was allowed to cool to 65° C., and bromine (23 ml, 448 mmol) dissolved in acetic acid (100 ml) was added drop wise over 30 minutes. The reaction was stirred for 3 days, and then filtered. The isolated crystals were washed with acetic acid (2×50 ml), water (2×100 ml), and acetic acid (1×50 ml) to afford the title compound.

Yield: 41.3 g, (79%).

HPLC-MS (Method B2): m/z=245 (M+); Rₜ=0.918 min.

General Procedure (A):

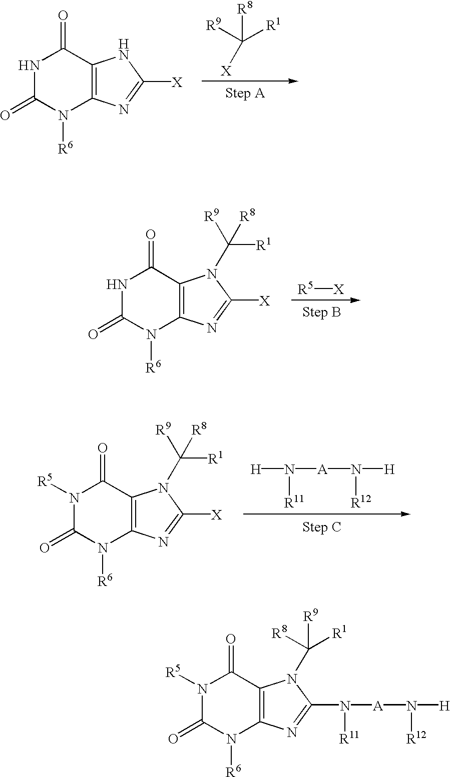

Step A:

The starting material (16 μmol) is dissolved in a mixture of DMF and DIEA (3% DIEA, 250 μl). The alkylation reagent R¹—CR⁹R⁹—X (16.8 μmol, 1.05 equiv) is dissolved in DMF (100 μl) and added. The mixture is heated to 65° C. for 2 h.

Step B:

Alkylation reagent R⁵—Br (32 μmol) is dissolved in DMF (100 μl) and added to the reaction mixture followed by a solution of TMG in DMF (1.16 ml TMG diluted to 5.8 ml, 48 μl). The mixture is kept at 65° C. for 4 h. Volatiles are stripped

Step C:

The diamine (200 μmol) is dissolved in a mixture of DMSO and DCHMA (3% DCHMA, 200 μl) and added to the reaction mixture. The reaction is kept at 50° C. for 44 h.

Samples are neutralized using HOAc (20 pi), stripped and purified by HPLC (Method A2).

General Procedure (B):

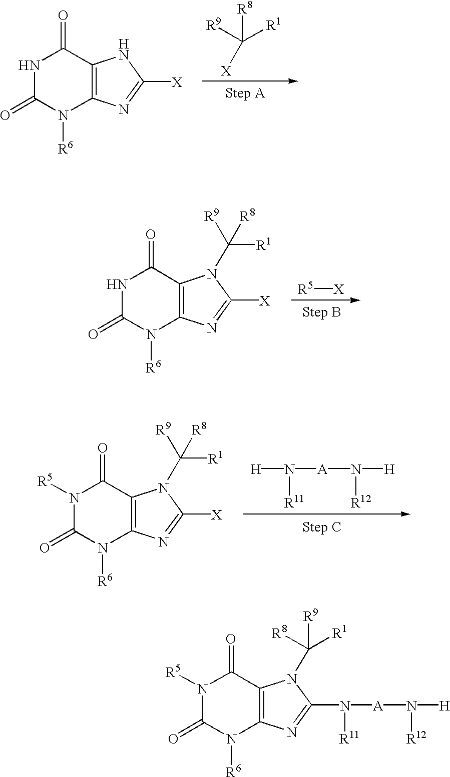

Step A:

The starting material (32 μmol) is dissolved in a mixture of DMF and DIEA (3% DIEA, 500 μl). The alkylation reagent R¹—CR⁹R⁹—X (33.6 μmol, 1.05 equiv) is dissolved in DMF (200 μl) and added. The mixture is heated to 65° C. for 2 h. Upon cooling to 25° C., K₂CO₃ (aq) is added (5.12M, 50 μL, 256 mmol). Volatiles are stripped.

Step B:

Alkylation reagent R⁵—Br (64 μmol) is dissolved in DMF (250 μl) and added to the reaction mixture. The mixture is kept at 25° C. for 48 h. Volatiles are stripped

Step C:

The diamine (400 μmol) is dissolved in DMSO and added to the reaction mixture. If the dihydrochloride salt of the diamine is employed, four equivalents of DCHMA are added. The reaction is kept at 50° C. for 48 h. Samples are neutralized using HOAc (30 μl), and purified by HPLC (Method A3).

General Procedure (C)

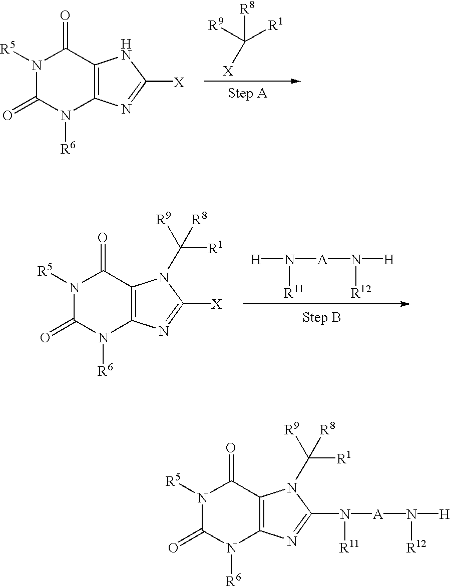

Step A:

The starting material (4.08 mmol) is dissolved in a mixture of DMF and DIEA (3% DIEA, 65 ml). The alkylation reagent R¹—CR⁹R⁹—X (4.28 mmol, 1.05 equiv) is dissolved in DMF (25.5 ml) and added. The mixture is heated to 65° C. for 2 h and poured onto ice followed by filtration of the alkylated product.

Step B:

Diamine (400 μmol) is dissolved in DMSO (400 μl) and added to the above product (32 mmol). The reaction is kept at 50° C. for 24-48 h. Samples are neutralized using HOAc (30 μl) and purified by HPLC (Method A2) or (Method A1)

General Procedure (D)

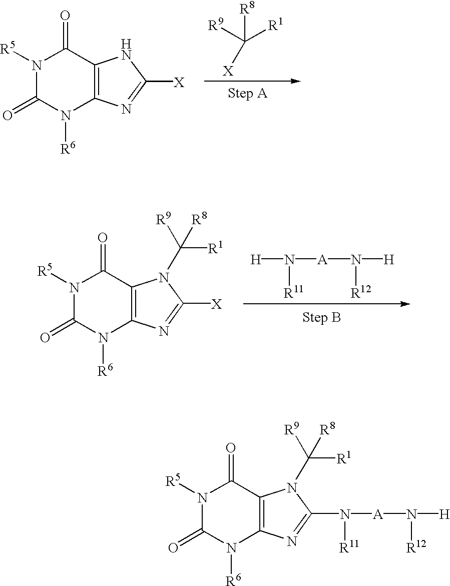

Step A:

The starting material (32 μmol) is dissolved in a mixture of DMF and DIEA (3% DIEA, 500 μl). The alkylation reagent R¹—CR⁹R⁹—X (33.6 μmol, 1.05 equiv) is dissolved in DMF (200 μl) and added. The mixture is heated to 65° C. for 2 h.

Step B:

Diamine (400 μmol) is dissolved in DMSO (400 μl) and added to the above reaction mixture. The reaction is kept at 50° C. for 48 h.

Samples are neutralized using HOAc (30 μl) and purified by HPLC (Method A2).

General Procedure (E):

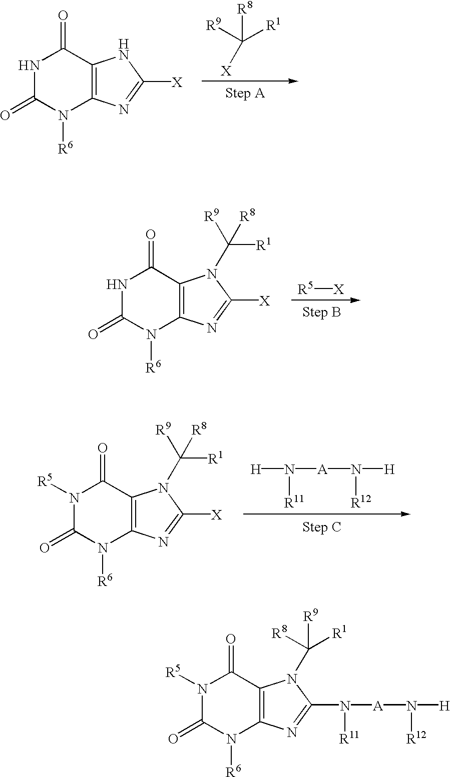

Step A:

The starting material (20.40 mmol) is dissolved in DMF (50 ml) and DIEA (10 mL). The alkylation reagent R¹—CR⁹R⁹—X (22.03 mmol, 1.08 equiv) is dissolved in DMF (10 ml) and added. Heating the mixture to 65° C. for 2 h affords the products that are isolated by filtration upon adding the reaction mixture onto ice (300 mL).

Step B:

The product from Step A (5.56 mmol) and alkylation reagent R⁵—Br (11.11 mmol) are dissolved in DMF (60 mL) and potassium carbonate is added to the reaction mixture. Upon stirring at 25° C. for 16 h the reaction mixture is poured onto ice (300 ml) and the product is isolated by filtration and dried in vacuo.

Step C:

The product from Step B (0.472 mmol) is dissolved in DMSO (5 ml) and the diamine (2.36 mmol) is added to the reaction mixture. If the dihydrochloride salt of the diamine is employed, K₂CO₃ (2.36 mmol) is added. The reaction is kept at 50° C. for 24 h and poured onto ice (20 ml). The product is isolated by filtration.

### EXAMPLES

### Example 1

### General Procedure (A)

(±) Cis-8-(2-Aminocyclohexylamino)-7-benzyl-3-methyl-1-(2-oxo-2-phenylethyl)-3,7-dihydropurine-2,6-dione

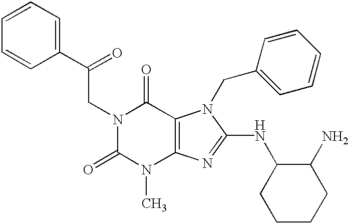

¹H NMR (DMSO-d₆): δ 8.10-8.01 (m, 2H); 7.82 (s br, 3H); 7.71 (t, 1H); 7.57 (t, 2H); 7.38-7.17 (m, 5H); 6.73 (d, 1H); 5.51-5.23 (m, 4H); 4.29-4.17 (m, 1H); 3.59 (s br, 1H); 3.42 (s, 3H); 1.89-1.29 (m, 8H). HPLC-MS (Method B1): m/z=487 (M+1); Rₜ=3.087 min

### Example 2

### General Procedure (A)

(±) Cis-8-(2-Aminocyclohexylamino)-7-(2-chlorobenzyl)-1-(2-hydroxy-2-phenylethyl)-3-methyl-3,7-dihydropurine-2,6-dione

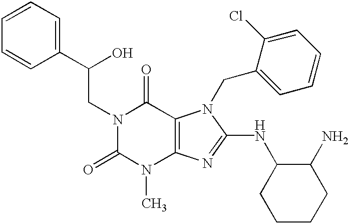

Styrene oxide was employed instead of R⁵—X

¹H NMR (DMSO-d₆): δ 7.79 (s br, 3H); 7.55-7,48 (m, 1H); 7,38-7,15 (m, 7H); 6,81-6,71 (m, 1H); 6,63-6,54 (m, 1H); 5.59-5.35 (m, 2H); 4.93-4.81 (m, 1H); 4.24 (s br, 1H); 4.14-4.04 (m, 1H); 3.41 (s, 3H); 1.86-1.29 (m, 8H). HPLC-MS (Method B1): m/z=523 (M+1); Rₜ=3.058 min.

### Example 3

### General Procedure (C)

8-(2-(S)-Aminocyclohexyl-(S)-amino)-7-(2-iodobenzyl)-3-methyl-3,7-dihydropurine-2,6-dione

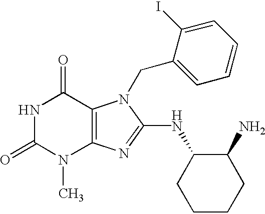

¹H NMR (DMSO-d₆): δ 10.68 (s, 1H); 9.92 (d, 1H); 7.85 (s br, 3H); 7.32 (t, 1H); 7.12-6.97 (m, 2H); 6.42 (d, 1H); 5.36-4.96 (dd, 2H); 3.86-3.68 (m, 1H); 3.36 (s, 3H); 3.09-2.93 (m, 1H) 2.08-1.12 (m, 8H). HPLC-MS (Method B1): m/z=495 (M+1); Rₜ=2.313 min.

### Example 4

### General Procedure (C)

8-(2-(R)-Aminocyclohexyl-(R)-amino)-7-(2-iodobenzyl)-3-methyl-3,7-dihydropurine-2,6-dione

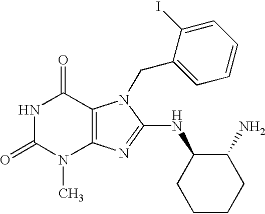

¹H NMR (DMSO-d₆): δ 10.68 (s, 1H); 7.92 (d, 1H); 7.85 (s br, 3H); 7.33 (t, 1H); 7.10-7.00 (m, 2H); 6.42 (m, 1H); 5.29 (d, 1H); 5.03 (d, 1H); 3.77 (m, 1H); 3.36 (s, 3H); 3.01 (m, 1H); 1.98 (m, 2H); 1.69 (m, 2H); 1.42 (m, 1H); 1.24 (m, 3H). HPLC-MS (Method B2): m/z=495 (M+1); Rₜ=3.70 min.

### Example 5

### General Procedure (C)

(±) Cis-8-(2-Aminocyclohexylamino)-7-(2-iodobenzyl)-3-methyl-3,7-dihydropurine-2,6-dione

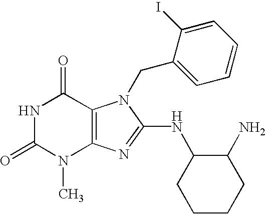

¹H NMR (DMSO-d₆): δ 10.67 (s, 1H); 7.91 (d, 1H); 7.76 (s br, 3H); 7.31 (t, 1H); 7.04 (t, 1H); 6.73 (d, 1H); 6.44 (d, 1H); 5.39-5.14 (m, 2H); 1.06 (s br, 1H); 3.59 (s br, 1H); 3.35 (s, 3H); 1.86-1-28 (m, 8H). HPLC-MS (Method B1): m/z=495 (M+1) Rₜ=2.313

### Example 6

### General Procedure (C)

8-(2-(S)-Aminocyclohexyl-(S)-amino)-7-biphenyl-2-ylmethyl-3-methyl-3,7-dihydropurine-2,6-dione

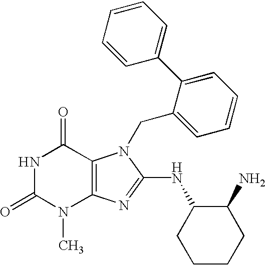

¹H NMR (DMSO-d₆): δ 10.58 (s, 1H); 7.87 (s br, 3H); 7.55-7.23 (m, 7H); 7.03 (d, 1H); 6.58 (d, 1H); 5.37 (d, 1H); 5.11 (d, 1H); 3.78 (m, 1H); 3.34 (s, 3H); 3.02 (m, 1H); 2.03 (m, 2H); 1.74 (m, 2H); 1.45 (m, 1H); 1.26 (m, 3H). HPLC-MS (Method B2): m/z=445 (M+1); Rₜ=4.03 min.

### Example 7

### General Procedure (C)

(±) Cis-8-(2-Aminocyclohexylamino)-7-biphenyl-2-ylmethyl-3-methyl-3,7-dihydropurine-2,6-dione

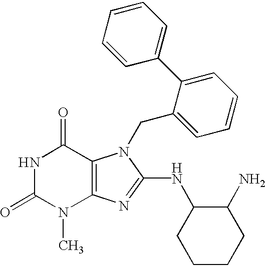

¹H NMR (DMSO-d₆): δ 10.57 (s, 1H); 7.79 (s br, 3H); 7.50-7.22 (m, 8H); 6.66 (d, 1H); 6.54 (d, 1H); 5.39 (d, 1H); 5.24 (d, 1H); 4.22 (m, 1H); 3.55 (m, 1H); 3.32 (s, 3H); 1.80-1.30 (m, 8H). HPLC-MS (Method B2): m/z=445 (M+1); Rₜ=3.92.

### Example 8

### General Procedure (C)

8-(2-(S)-Aminocyclohexyl-(S)-amino)-7-(2-bromobenzyl)-3-methyl-3,7-dihydropurine-2,6-dione

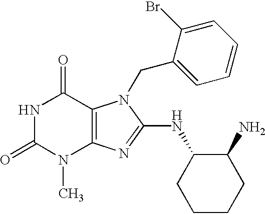

¹H NMR (DMSO-d₆): δ 10.68 (s, 1H); 7.87 (s br, 3H); 7.69 (d, 1H); 7.37-7.19 (m, 2H); 7.045 (d, 1H); 6.51 (d, 1H); 5.46-5.08 (dd, 2H); 3.87-3.71 (m, 1H); 3.36 (s, 3H); 3.10-2.92 (m, 1H); 2.09-1.09 (m, 8H). HPLC-MS (Method B1): m/z=449 (M+1); Rₜ=1.932 min.

### Example 9

### General procedure (C)

(±) Cis-8-(2-Aminocyclohexylamino)-7-(2-bromobenzyl)-3-methyl-3,7-dihydropurine-2,6-dione

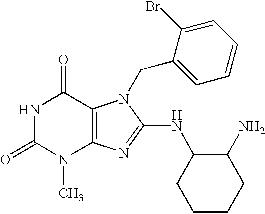

¹H NMR (DMSO-d₆): δ 10.67 (s, 1H); 7.77 (s br, 3H); 7.67 (d, 1H); 7.36-7.17 (m, 2H); 6.74 (d, 1H); 5.51-5.26 (dd, 2H); 4.22 (s br, 1H); 3.58 (s br, 1H); 3.35 (s, 3H); 1.87-1.28 (m, 8H).

HPLC-MS (Method B1): m/z=449 (M+1); Rₜ=1.926

### Example 10

### General Procedure (C)

8-(2-(S)-Aminocyclohexyl-(S)-amino)-7-(2-chlorobenzyl)-3-methyl-3,7-dihydropurine-2,6-dione

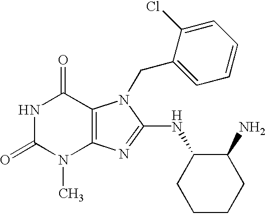

¹H NMR (DMSO-d₆): δ 10.68 (s br, 1H); 7.86 (s br, 3H); 7.56-7.48 (m, 1H); 7.37-7.22 (m, 2H); 7.10-6.99 (m, 1H); 6.61-6.52 (m, 1H) 1.51-5.15 (dd, 2H); 3.86-3.69 (m. 1H); 3.36 (s, 3H); 3.08-2.93 (m, 1H); 2.09-1.12 (m, 8H). HPLC-MS (Method B1): m/z=403 (M+1); Rₜ 2.184 min.

### Example 11

### General Procedure (C)

8-(2-(R)-Aminocyclohexyl-(R)-amino)-7-(2-chlorobenzyl)-3-methyl-3,7-dihydropurine-2,6-dione

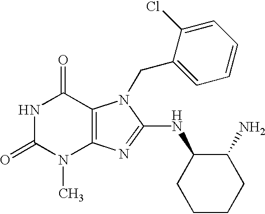

¹H NMR (DMSO-d₆): δ 10.68 (s, 1H); 7.92 (s br, 3H); 7.52 (d, 1H); 7.30 (t+t, 2H); 7.08 (d, 1H); 6.57 (d, 1H); 5.44 (d, 1H); 5.21 (d, 1H); 3.77 (m, 1H); 3.36 (s, 3H); 3.02 (m, 1H); 2.00 (m, 2H); 1.68 (m, 2H); 1.42 (m, 1H); 1.23 (m, 3H).

### Example 12

### General Procedure (C)

(±) Cis-8-(2-Aminocyclohexylamino)-7-(2-chlorobenzyl)-3-methyl-3,7-dihydropurine-2,6-dione

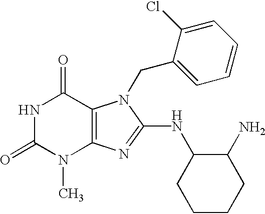

¹H NMR (DMSO-d₆): δ 10.68 (s br, 1H); 7.75 (s br, 3H); 7.505 (dd, 1H); 7.35-7.22 (m, 2H); 7.76-6.58 (m, 2H); 5.52-5.33 (dd, 2H); 4.22 (s br, 1H); 3.58 (s, 1H); 3.14 (s, 3H); 1.87-1.27 (m, 8H). HPLC-MS (Method B1): m/z=403 (M+1); Rₜ=2.192 min.

### Example 13

### General Procedure (A)

(±) Cis-8-(2-Aminocyclohexylamino)-1,7-bis-(2-chlorobenzyl)-3-methyl-3,7-dihydropurine-2,6-dione

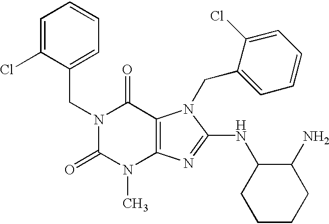

¹H NMR (DMSO-d₆): δ 7.79 (s br, 3H); 7.50-7.37 (m, 2H); 7.35-7.10 (m, 4H); 6.86 (d, 1H); 6.77 (d, 1H); 5.58 (d, 1H); 5.46 (dd, 2H); 4.99 (s, 2H); 4.27 (m, 1H); 3.60 (m, 1H); 3.46 (s, 3H); 1.80-1.30 (m, 8H). (Method B2): m/z=527 (M+1); Rₜ=5.12 min.

### Example 14

### General Procedure (A)

(±) Cis-2-[8-(2-Aminocyclohexylamino)-7-(2-chlorobenzyl)-3-methyl-2,6-dioxo-1,2,3,6-tetrahydropurin-1-ylmethyl]benzonitrile

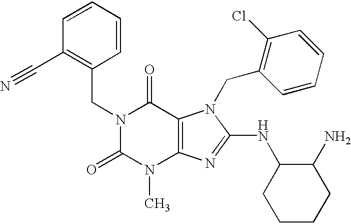

¹H NMR (DMSO-d₆): δ 7.80 (s+d, 4H); 7.57 (t, 1H); 7.50 (d, 1H); 7.41 (t, 1H); 7.29 (t+t, 2H); 7.09 (d, 1H); 6.86 (d, 1H); 6.68 (d, 1H); 5.48 (dd, 2H); 5.12 (s, 2H); 4.26 (m, 1H); 3.60 (m, 1H); 3.44 (s, 3H); 1.80-1.35 (m, 8H). (Method B2): m/z=518 (M+1); Rₜ=4.72 min.

### Example 15

### General Procedure (A)

(±) Cis-8-(2-Aminocyclohexylamino)-7-(2-chlorobenzyl)-3-methyl-1-(2-oxo-2-phenylethyl)-3,7-dihydropurine-2,6-dione

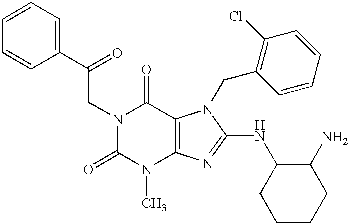

¹H NMR (DMSO-d₆): δ 8.01 (d, 2H); 7.77 (s br, 3H); 7.69 (t, 1H); 7.55 (t, 2H); 7.49 (d, 1H); 7.29 m, 2H); 6.86 (d, 1H); 6.69 (d, 1H); 5.46 (dd, 2H); 5.25 (dd, 2H): 4.28 (m, 1H); 3.64 (m, 1H); 3.46 (s, 3H); 1.80-1.30 (m, 8H). (Method B2): m/z=521 (M+1); Rₜ=4.85 min.

### Example 16

### General Procedure (E)

8-(2-(R)-Aminocyclohexyl-(S)-amino)-7-(2-chlorobenzyl)-3-methyl-1-(2-oxo-2-phenylethyl)-3,7-dihydropurine-2,6-dione

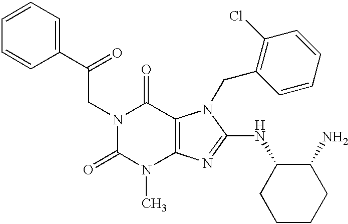

The enantiomerically pure compound was isolated using (Method D).

¹H NMR (DMSO-d₆): δ 8.01 (d, J=7.54 Hz, 2H); 7.83 (s, 3H); 7.70 (m, 1H); 7.53 (m, 3H); 7.30 (m, 2H); 6.92 (d, J=6.41 Hz, 1H); 6.67 (d, J=5.28 Hz, 1H); 5.51 (d, J=18.09 Hz, 1H); 5.43 (d, J=18.09 Hz, 1H); 5.29 (d, J=18.00 Hz, 1H); 5.22 (d, J=18.00 Hz, 1H); 4.28 (s, 1H); 3.63 (s, 1H); 3.46 (s, 3H); 1.67 (m, 6H); 1.40 (s, 2H).

### Example 17

### General Procedure (A)

(±) Cis-8-(2-Aminocyclohexylamino)-7-(2-chlorobenzyl)-3-methyl-1-phenethyl-3,7-dihydropurine-2,6-dione

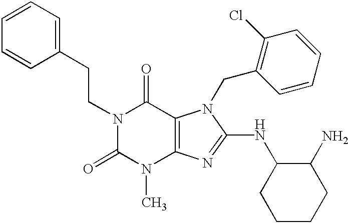

¹H NMR (DMSO-d₆): δ 7.78 (s br, 3H); 1.52 (d, 1H); 7.35-7.24 (m, 4H); 7.24-7.12 (m, 3H); 6.79 (d, 1H); 6.61 (d, 1H); 5.47 (dd, 2H); 4.24 (m, 1H); 3.94 (t, 2H); 3.59 (m, 1H); 3.43 (s, 3H); 2.73 (t 1H); 1.80-1.30 (m, 8H). (Method B2): m/z=507 (M+1); Rₜ=5.10 min.

### Example 18

### General Procedure (A)

(±) Cis-8-(2-Aminocyclohexylamino)-7-(2-bromobenzyl)-1-(2-chlorobenzyl)-3-methyl-3,7-dihydropurine-2,6-dione

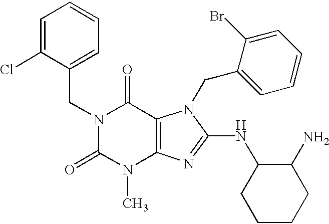

¹H NMR (DMSO-d₆): δ 7.76 (s br, 3H); 7.66 (d, 1H); 7.42 (d, 1H); 7.40-7.15 (m, 4H); 6.87 (d, 1H); 6.77 (d, 1H); 6.62 (d, 1H); 5.41 (dd, 2H); 4.98 (s, 2H); 4.27 (m, 1H); 3.61 (m, 1H); 3.46 (s, 3H); 1.80-1.35 (m, 8H). (Method B2): m/z=573 (M+1); Rₜ=5.37 min

### Example 19

### General Procedure (A)

(±) Cis-2-[8-(2-Aminocyclohexylamino)-7-(2-bromobenzyl)-3-methyl-2,6-dioxo-1,2,3,6-tetrahydropurin-1-ylmethyl]benzonitrile

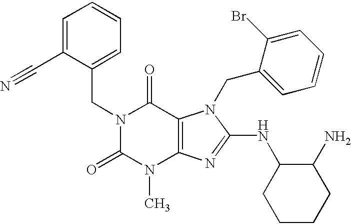

¹H NMR (DMSO-d₆): W7.78 (d, 1H); 7.74 (s br, 3H); 7.67 (d, 1H); 7.57 (t, 1H); 7.41 (t, 1H); 7.31 (t, 1H); 7.22 (t, 1H); 7.09 (d, 1H); 6.86 (d, 1H); 6.61 (d, 1H); 5.42 (dd, 2H); 5.11 (s, 2H); 4.26 (m, 1H); 3.61 (m, 1H); 3.45 (s, 3H); 1.80-1.35 (m, 8H). (Method B2): m/z=562 (M+1); Rₜ=4.88

### Example 20

### General Procedure (A)

(±) Cis-8-(2-Aminocyclohexylamino)-7-(2-bromobenzyl)-3-methyl-1-(2-oxo-2-phenylethyl)-3,7-dihydropurine-2,6-dione

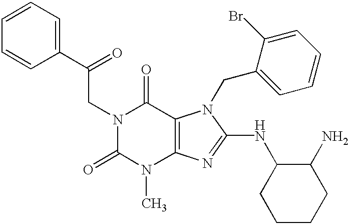

¹H NMR (DMSO-d₆): δ 8.01 (d, 2H); 7.74 (s br, 3H); 7.67 (m, 2H); 7.55 (m, 2H); 7.32 (t, 1H); 7.25 (t, 1H); 6.88 (d, 1H); 6.61 (d, 1H); 5.41 (dd, 2H); 5.25 (dd, 2H); 4.28 (m, 1H); 3.63 (m, 1H); 3.46 (s, 3H); 1.80-1.35 (m, 8H). (Method B2): m/z=567 (M+1); Rₜ=5.02 min.

### Example 21

### General Procedure (A)

(±) Cis-8-(2-Aminocyclohexylamino)-7-(2-bromobenzyl)-3-methyl-1-phenethyl-3,7-dihydropurine-2,6-dione

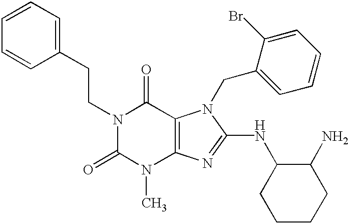

¹H NMR (DMSO-d₆): δ 7.75 (s br, 3H); 7.69 (d, 1H); 7.35-7.10 (m, 7H); 6.80 (d, 1H); 6.54 (d, 1H); 5.43 (dd, 2H); 4.23 (m, 1H); 3.94 (t, 2H); 3.61 (m, 1H); 3.43 (s, 3H); 2.73 (2H); 1.80-1.30 (m, 8H). (Method B2): m/z=551 (M+1); Rₜ=5.28 min.

### Example 22

### General Procedure (D)

(±) Cis-8-(2-Aminocyclohexylamino)-3-methyl-7-(2-methylbenzyl)-3,7-dihydropurine-2,6-dione

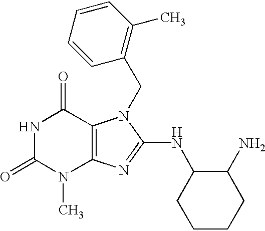

HPLC-MS (Method A3): m/z=383 (M+1); Rₜ=3.10 min.

### Example 23

### General Procedure (C)

8-(2-(S)-Aminocyclohexyl-(S)-amino)-1,3-dimethyl-7-(2-methylbenzyl)-3,7-dihydropurine-2,6-dione

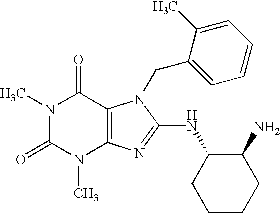

¹H NMR (MeOH-d4): δ 7.16 (m, 4H), 6.47 (d 1H), 5.36 (dd, 2H), 3.98 (m, 1H), 3.54 (s, 3H), 3.22 (s, 3H), 3.09 (m, 1H), 2.40 (s, 3H), 1.20-2.34 (m, 10H)HPLC-MS (Method B1) m/z=397 (M+1); Rₜ=2.15 min

### Example 24

### General Procedure (D)

(±) C is 8-(2-Aminocyclohexylamino)-1,3-dimethyl-7-(2-methylbenzyl)-3,7-dihydropurine-2,6-dione

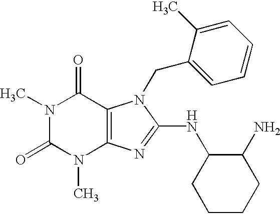

HPLC-MS (Method A3): m/z=397 (M+1); Rₜ=3.50 min.

### Example 25

### General Procedure (D)

(±) Cis-8-(2-Aminocyclohexylamino)-7-(2-chlorobenzyl)-3-methyl-3,7-dihydropurine-2,6-dione

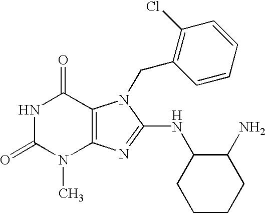

HPLC-MS (Method A3): m/z=403 (M+1); Rₜ=3.10 min.

### Example 26

### General Procedure (D)

(±) Cis-8-(2-Aminocyclohexylamino)-7-(2,5-difluorobenzyl)-3-methyl-3,7-dihydropurine-2,6-dione

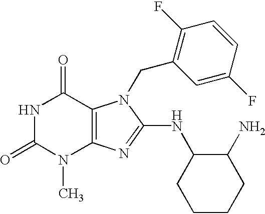

HPLC-MS (Method A3): m/z=405 (M+1); Rₜ=3.30 min.

### Example 27

### General Procedure (C)

(±) Cis 2-[8-(2-Aminocyclohexylamino)-1,3-dimethyl-2,6-dioxo-1,2,3,6-tetrahydropurin-7-ylmethyl]benzonitrile

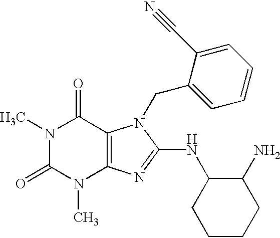

¹H NMR (MeOH-d4): δ 7.77(d, 1H), 7.60(t, 1H), 7.41 (t, 1H), 7.06(d, 1H), 5.61(m, 3H), 4.37(s, 1H), 3.79(s, 1H), 3.50(m, 3H), 3.23(m, 4H), 1.62(m, 9H). HPLC-MS (Method B1) m/z=408 (M+1); Rₜ=1.78 min.

### Example 28

### General Procedure (C)

2-[8-(2-(S)-Aminocyclohexyl-(S)-amino)-1,3-dimethyl-2,6-dioxo-1,2,3,6-tetrahydropurin-7-ylmethyl]benzonitrile

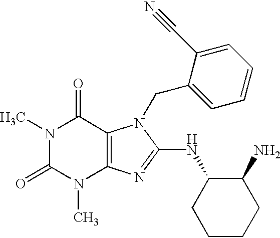

¹H NMR (MeOH-d4): δ 7.53 (m, 4H), 7.00 (d, 1H), 5.58 (dd, 2H), 3.99 (m, 1H), 3.52 (s, 3H), 3.21 (s, 3H), 3.12 (m, 1H), 1.20-2.22 (m, 9H). HPLC-MS (Method B1) m/z=408 (M+1); Rₜ 1.84 min

### Example 29

(±) Cis-2-[8-(2-Aminocycloheptylamino)-3-methyl-2,6-dioxo-1,2,3,6-tetrahydropurin-7-ylmethyl]benzonitrile. TFA

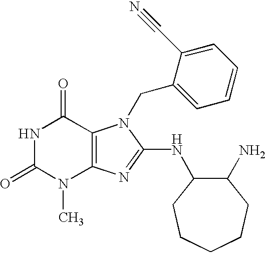

Step A: 2-(8-Bromo-3-methyl-2,6-dioxo-1,2,3,6-tetrahydropurin-7-ylmethyl)benzonitrile (29A)

8-Bromo-3-methyl-3,7-dihydropurine-2,6-dione (2,5 g, 10.2 mmol), dimethyl formamide (30 ml), 2-cyanobenzylbromid (2.15 g, 11.0 mmol), and diisopropylethylamine (5 ml) were stirred at 65° C. for two days. The solvents were evaporated and the remaining was stirred with ethyl acetate (150 ml) and water (150 ml) for 30 minutes. The precipitate was collected by filtration to afford compound 29A as white crystals.

Yield: 3.20 g (87%).

HPLC-MS (Method B1): m/z=360 (M+), Rₜ=2,54 min.

Step B: (±) Cis-2-[8-(2-Aminocycloheptylamino)-3-methyl-2,6-dioxo-1,2,3,6-tetrahydropurin-7-ylmethyl]benzonitrile. TFA (29)

2-(8-Bromo-3-methyl-2,6-dioxo-1,2,3,6-tetrahydropurin-7-ylmethyl)benzonitrile (29A) (204 mg, 0.57 mmol) and potassium carbonate (391 mg, 2.83 mmol) were dissolved in DMSO (2 ml), and cis-cycloheptane-1,2-diamine (180 mg, 1.4 mmol) was added. The mixture was stirred at 65° C. for four days, and then poured into water (20 ml) and dichloromethane (30 ml). The layers were separated and the aqueous layer was extracted with dichloromethane (2×30 ml). The combined organic layers were washed with water, dried with sodium sulphate, filtered and evaporated. The crude product was purified by preparative HPLC (method A1, Rt=7.27 min.) to give the title compound as a clear oil.

Yield: 53 mg (18%).

¹H NMR (300 MHz, DMSO-d₆): δ 1.3-1.9 (m, 10H); 3.3 (s, 3H); 3.5 (s br, 1H); 4.4 (m, 1H); 5.5 (s, 2H); 6.7 (d, 1H); 6.8 (d, 1H); 7.5 (t, 1H); 7.6 (t, 1H); 7.7 (s br, 3H), 7.9 (d, 1H); 10.7 (s, 1H). HPLC-MS (Method B1) m/z=408.3 (M+1); Rₜ=1.97 min.

### Example 30

(±) Cis-8-(2-Aminocycloheptylamino)-7-(2-chlorobenzyl)-3-methyl-3,7-dihydropurine-2,6-dione. TFA

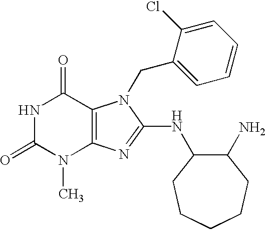

Step A: 8-Bromo-7-(2-chlorobenzyl)-3-methyl-3,7-dihydropurine-2,6-dione (30A)

Compound 30A was prepared as described in the General procedure C, step A. HPLC-MS (Method B2) m/z=371 (M+2); Rₜ=3.031 min.

Step B: (±) Cis-8-(2-Aminocycloheptylamino)-7-(2-chlorobenzyl)-3-methyl-3,7-dihydropurine-2,6-dione. TFA (30)

8-Bromo-7-(2-chlorobenzyl)-3-methyl-3,7-dihydropurine-2,6-dione (30A) (201 mg, 0.54 mmol) and cis-cycloheptane-1,2-diamine (139 mg, 1.1 mmol) were reacted and purified as described in example 29, step B, to afford the title compound as white crystals.

Yield: 37 mg (16%).

Prep. HPLC (method A1): Rₜ=7.63 min.

¹H NMR (DMSO-d₆): δ 1.3-1.4 (m, 10H); 3.3 (s, 3H), 3.5 (s br, 1H); 4.4 (m, 1H); 5.4 (2 d, 2H); 6.6 (dd, 1H); 6.7 (d, 1H); 7.3 (dq, 2H); 7.5 (dd, 1H), 7.7 (s br, 3H), 10.7 (s, 1H). HPLC-MS (Method B1) m/z=417.1 (M+1); Rₜ=2.34 min.

### Example 31

### General Procedure (C)

8-(2-(S)-Aminocyclohexyl-(S)-amino)-7-(2-chlorobenzyl)-1,3-dimethyl-3,7-dihydropurine-2,6-dione

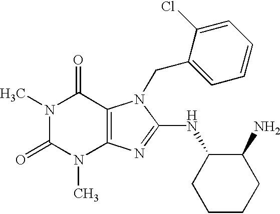

### Example 32

### General Procedure (D)

(±) Cis 8-(2-Aminocyclohexylamino)-7-(2-chlorobenzyl)-1,3-dimethyl-3,7-dihydropurine-2,6-dione

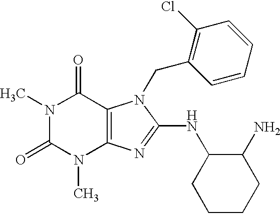

HPLC-MS (Method A3): m/z=417 (M+1); Rₜ=3.60 min.

### Example 33

### General Procedure (D)

(±) Cis-8-(2-Aminocyclohexylamino)-7-(2,3-difluorobenzyl)-1,3-dimethyl-3,7-dihydropurine-2,6-dione

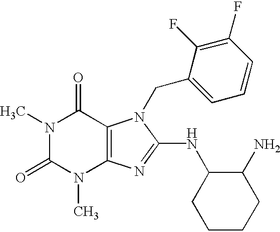

HPLC-MS (Method A3): m/z=419 (M+1); Rₜ=3.30 min.

### Example 34

(±) Cis-2-[8-(2-Aminocycloheptylamino)-1,3-dimethyl-2,6-dioxo-1,2,3,6-tetrahydropurin-7-ylmethyl]benzonitrile. TFA

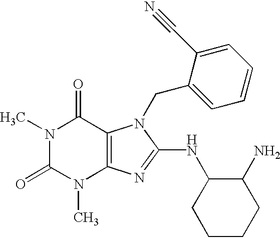

Step A: 2-(8-Chloro-1,3-dimethyl-2,6-dioxo-1,2,3,6-tetrahydropurin-7-ylmethyl)benzonitrile (34A)

Compound 34A was prepared as described in the General procedure C, step A.

HPLC-MS (Method B1) m/z=330 (M+1); Rₜ=2.93 min.

Step B: (±) Cis-2-[8-(2-Aminocycloheptylamino)-1,3-dimethyl-2,6-dioxo-1,2,3,6-tetrahydropurin-7-ylmethyl]benzonitrile. TFA (34)

2-(8-Chloro-1,3-dimethyl-2,6-dioxo-1,2,3,6-tetrahydropurin-7-ylmethyl)benzonitrile (34A) (205 mg, 0.62 mmol) and cis-cycloheptane-1,2-diamine (159 mg, 1.2 mmol) were reacted and purified as described in example 29, step B, to afford the title compound as white crystals.

Yield: 111 mg (42%).

Prep. HPLC (method A1): Rₜ=7.67 min.

¹H NMR (DMSO-d₆): δ 1.3-1.9 (m, 10H); 3.1 (s, 3H), 3.4 (s, 3H); 3.5 (s br, 1H); 4.4 (m, 1H); 5.6 (s, 2H); 6.8 (dd, 2H); 7.5 (dd, 1H); 7.6 (ddd, 1H); 7.5 (dd, 1H), 7.8 (s br, 3H), 7.9 (dd, 1H).

HPLC-MS (Method B1) m/z=422.2 (M+1); Rₜ=2.16 min.

### Example 35

(±) Cis-8-(2-Aminocycloheptylamino)-7-(2-chlorobenzyl)-1,3-dimethyl-3,7-dihydropurine-2,6-dione. TFA

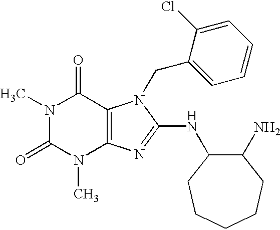

Step A: 8-Chloro-7-(2-chlorobenzyl)-1,3-dimethyl-3,7-dihydropurine-2,6-dione (35A)

Compound 35A was prepared as described in the General procedure C, step A.

HPLC-MS (Method B1) m/z=339 (M+); Rₜ=3.95 min.

Step B: (±) Cis-8-(2-Aminocycloheptylamino)-7-(2-chlorobenzyl)-1.3-dimethyl-3,7-dihydropurine-2,6-dione. TFA (35)

8-Chloro-7-(2-chlorobenzyl)-1,3-dimethyl-3,7-dihydropurine-2,6-dione (35A) (200 mg, 0.59 mmol) and cis-cycloheptane-1,2-diamine (151 mg, 1.2 mmol) were reacted and purified as described in example 29, step B, to afford the title compound as white crystals.

Yield: 31 mg (11%).

Prep. HPLC (method A1): Rₜ=8.25 min.

¹H NMR (DMSO-d₆): δ 1.3-1.9 (m, 10H); 3.1 (s, 3H), 3.4 (s, 3H); 3.5 (s br, 1H); 4.4 (m, 1H); 5.4 (2 d, 2H); 6.6 (dd, 1H); 6.7 (d, 1H); 7.3 (2 dd, 2H); 7.5 (d, 1H); 7.7 (s br, 3H). HPLC-MS (Method B1) m/z=431.2 (M+1); Rₜ=2.49 min.

### Example 36

### General Procedure (D)

(±) Cis-8-(2-Aminocyclohexylamino)-7-(2-difluoromethoxybenzyl)-3-methyl-3,7-dihydropurine-2,6-dione

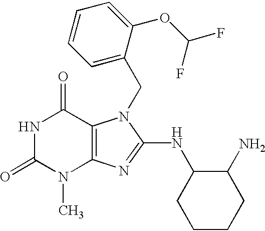

HPLC-MS (Method A3): m/z=435 (M+1); Rₜ=3.30 min.

### Example 37

### General Procedure (D)

(±) Cis-8-(2-Aminocyclohexylamino)-7-(2-difluoromethoxybenzyl)-1,3-dimethyl-3,7-dihydropurine-2,6-dione

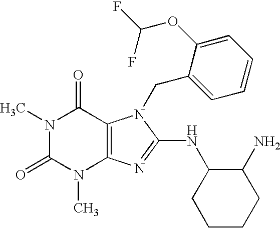

HPLC-MS (Method A3): m/z=448 (M+1); Rₜ=3.30 min.

### Example 38

### General Procedure (C)

8-(2-(S)-Aminocyclohexyl-(S)-amino)-1,3-dimethyl-7-(2-trifluoromethylbenzyl)-3,7-dihydropurine-2,6-dione

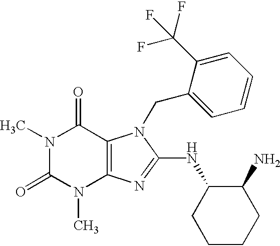

¹H NMR (MeOH-d4): δ 7.72 (m, 1H), 7.49 (m, 3H), 6.73 (d, 1H), 5.63 (dd, 2H), 3.98 (m, 1H), 3.54 (s, 3H), 3.16 (m, 4H), 1.22-2.23 (m, 1 OH), HPLC-MS (Method B1) m/z=451 (M+1); Rₜ=2.16 min

### Example 39

### General Procedure (C)

(±) Cis 8-(2-Aminocyclohexylamino)-1,3-dimethyl-7-(2-trifluoromethylbenzyl)-3,7-dihydropurine-2,6-dione

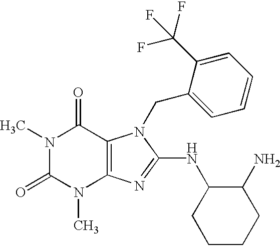

¹H NMR (MeOH-d4): δ 7.62 (d, 4H), 6.76 (m, 1H), 5.84 (d, 1H), 5.61 (d, 1H), 4.39 (m, 1H), 3.73 (m, 1H), 3.52 (s, 3H), 3.20 (s, 3H), 1.64 (m, 1 OH). HPLC-MS (Method B1) m/z=451 (M+1); Rₜ=4.09 min

### Example 40

### General Procedure (C)

8-(2-(S)-Aminocyclohexyl-(S)-amino)-7-(2-bromobenzyl)-1,3-dimethyl-3,7-dihydropurine-2,6-dione

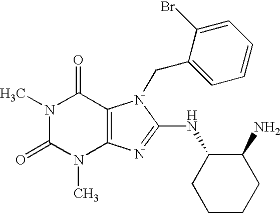

### Example 41

### General Procedure (C)

(±) Cis 8-(2-Aminocyclohexylamino)-7-(2-bromobenzyl)-1,3-dimethyl-3,7-dihydropurine-2,6-dione

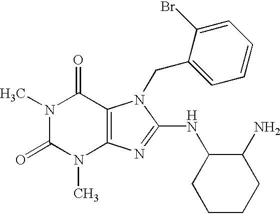

¹H NMR (MeOH-d4): δ 7.62 (d, 1H), 7.22 (m, 2H), 6.71 (d, 1H), 5.51 (dd, 2H), 4.36 (m, 1H), 3.74 (m, 1H), 3.51 (s, 3H), 3.19 (s, 3H), 1.62 (m, 9H). HPLC-MS (Method B1) m/z=462 (M+1); Rₜ=2.19 min

### Example 42

### General Procedure (D)

(±) Cis-8-(2-Aminocyclohexylamino)-7-(2-bromobenzyl)-1,3-dimethyl-3,7-dihydropurine-2,6-dione

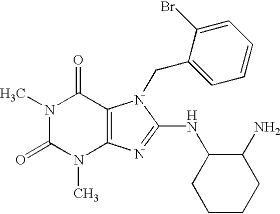

HPLC-MS (Method A3): m/z=461 (M+1); Rₜ=3.60 min.

### Example 43

### General Procedure (D)

(±) Cis-8-(2-Aminocyclohexylamino)-3-benzyl-7-(2-chlorobenzyl)-3,7-dihydropurine-2,6-dione

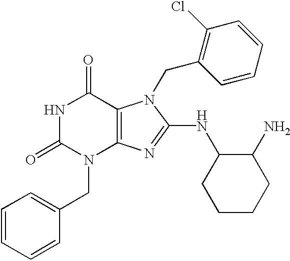

HPLC-MS (Method A3): m/z=478 (M+1); Rₜ=3.60 min.

### Example 44

### General Procedure (E)

(±) Cis-2-[8-(2-Aminocyclohexylamino)-7-(2-cyanobenzyl)-3-methyl-2,6-dioxo-1,2,3,6-tetrahydropurin-1-ylmethyl]benzonitrile

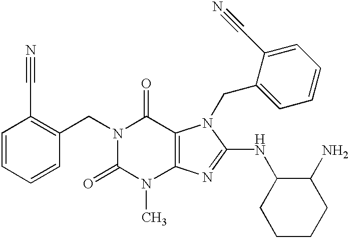

¹H NMR (DMSO-d₆): δ 7.89 (d, 1H); 7.79 (d, 1H); 7.64 (t, 1H); 7.57 (t, 1H); 7.47 (t, 1H); 7.41 (t, 1H); 7.13 (d, 1H); 6.84 (d, 1H); 5.65 (s, 2H); 5.11 (s, 2H); 3.92 (m, 1H); 3.41 (s, 3H); 3.12 (m, 1H); 1.80-1.15 (m, 8H). HPLC-MS (Method B1) m/z=509 (M+1) 531 (M+23); Rₜ=2.527 min.

### Example 45

### General Procedure (E)

8-(2-(S)-Aminocyclohexyl-(S)-amino)-7-(2-chlorobenzyl)-3-methyl-1-(2-oxo-2-phenylethyl)-3,7-dihydropurine-2,6-dione

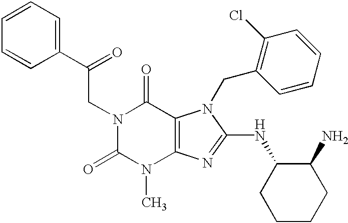

¹H NMR (DMSO-d₆): δ 8.03-7.77 (m, 4H); 7.68-7.55 (m, 1H); 7.53-7.36 (m, 3H); 7.29-7.09 (m, 3H); 6.59-6.46 (m, 1H); 5.45-5.23 (1H); 5.23-5.08 (m, 3H); 3.88-3.64 (m, 2H); 3.38 (s, 3H); 3.05-2.86 (m, 1H); 2.03-1.79 (m, 2H); 1.73-1.52 (m, 2H). HPLC-MS (Method B1) m/z=521(M+1); Rₜ=2.967 min.

### Example 46

### General Procedure (D)

(±) Cis-8-(2-Aminocyclohexylamino)-3-benzyl-7-(2-bromobenzyl)-3,7-dihydropurine-2,6-dione

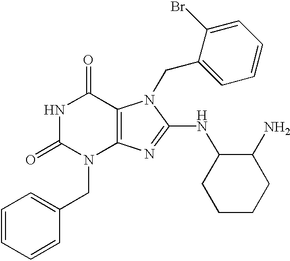

HPLC-MS (Method A3): m/z=523 (M+1); Rₜ=4.00 min.

### Example 47

### General Procedure (B))

(±) Cis-8-(2-Aminocyclohexylamino)-7-(2-chlorobenzyl)-3-methyl-1-(2-oxo-2-thiophen-3-yl-ethyl)-3,7-dihydropurine-2,6-dione

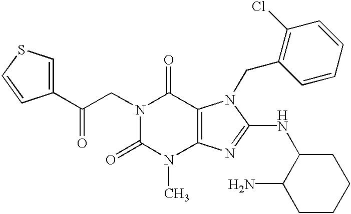

HPLC-MS (Method A3): m/z=528 (M+1); Rₜ=4.20 min.

### Example 48

### General Procedure (B)

2-(8-(2-(S)-Aminocyclohexyl-(S)-amino)-1-[2-(3-fluorophenyl)-2-oxoethyl]-3-methyl-2,6-dioxo-1,2,3,6-tetrahydropurin-7-ylmethyl)benzonitrile

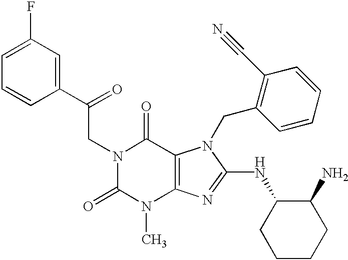

HPLC-MS (Method A3): m/z=530 (M+1); Rₜ=4.00 min.

### Example 49

### General Procedure (E)

8-(2-(S)-Aminocyclohexyl-(S)-amino)-7-(2-bromobenzyl)-3-methyl-1-(2-oxo-2-phenylethyl)-3,7-dihydropurine-2,6-dione

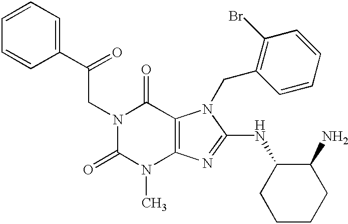

HPLC-MS (Method B1) m/z=565 (M+1); Rₜ=3,23 min.

### Example 50

### General Procedure (B)

8-(2-(S)-Aminocyclohexyl-(S)-amino)-7-(2-bromobenzyl)-3-methyl-1-(2-oxo-2-thiophen-3-yl-ethyl)-3,7-dihydropurine-2,6-dione

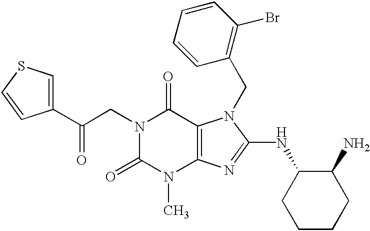

¹H NMR (DMSO-d₆): δ 8.64-8.68 (m, 1H), 7.64-7.72 (m, 2H), 7.48-7.54 (d, 1H), 7.28-7.36 (t, 1H), 7.18-7.26 (t, 1H), 6.48-6.52 (d, 1H), 6.35 (s, 2H), 5.14 (s, 2H), 1.06-2.00 (m, 8H). HPLC-MS (Method B2) m/z=573 (M+1); Rₜ=5.00 min.

### Example 51

### General Procedure (B)

(±) Cis-8-(2-Aminocyclohexylamino)-7-(2-bromobenzyl)-3-methyl-1-(2-oxo-2-thiophen-3-yl-ethyl)-3,7-dihydropurine-2,6-dione

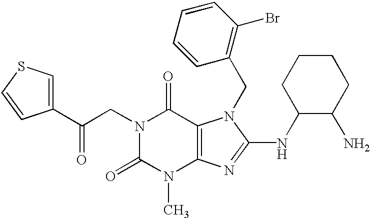

HPLC-MS (Method A3): m/z=572 (M+1); Rₜ=4.20 min.

### Example 52

8-(2-(S)-Aminocyclohexyl-(S)-amino)-7-(2-bromobenzyl)-3-methyl-1-phenethyl-3,7-dihydropurine-2,6-dione. TFA

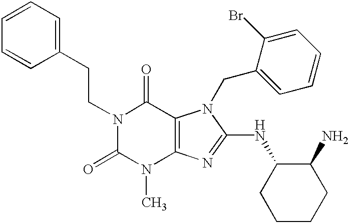

Step A: 8-Bromo-7-(2-bromobenzyl)-3-methyl-3,7-dihydropurine-2,6-dione (52A)

8-Bromo-3-methyl-3,7-dihydropurine-2,6-dione (5 g, 20.4 mmol), dimethyl formamide (150 ml), 2-bromobenzylbromid (5.35 g, 21.4 mmol), and diisopropylethylamine (7 ml) were reacted and purified as described in example 29, step A, to afford compound 52A as white crystals.

Yield: 7 g (83%).

HPLC-MS (Method B2): m/z=415 (M+1), Rₜ=3.129 min.

Step B: 8-Bromo-7-(2-bromobenzyl)-3-methyl-1-phenethyl-3,7-dihydropurine-2,6-dione (52B)

8-Bromo-7-(2-bromobenzyl)-3-methyl-3,7-dihydropurine-2,6-dione (52A) (2,0 g, 4.8 mmol), dimethyl formamide (50 ml), 2-bromoethylbenzen (1.92 g, 9.7 mmol), and potassium carbonate (2,0 g, 14.5 mmol) were stirred at 50° C. for 20 hours. The mixture was poured into water (250 ml) and ethyl acetate (20 ml). The precipitate was collected by filtration to afford compound 52B as white crystals.

Yield: 2.32 g (93%).

HPLC-MS (Method B2): m/z=519 (M+1), Rₜ=5.06 min.

Step C: 8-(2-(S)-Aminocyclohexyl-(S)-amino)-7-(2-bromobenzyl)-3-methyl-1-phenethyl-3,7-dihydropurine-2,6-dione. TFA (52)

8-Bromo-7-(2-bromobenzyl)-3-methyl-1-phenethyl-3,7-dihydropurine-2,6-dione (52B) (250 mg, 0.48 mmol) and (1S, 2S)-(+)-1,2-diamino-cyclohexan (277 mg, 2.41 mmol) were dissolved in DMSO (10 ml). The mixture was stirred at 65° C. for two days, and then poured into water (100 ml) and dichloromethane (100 ml). The layers were separated and the aqueous layer was extracted with dichloromethane (2×100 ml). The combined organic layers were washed with water, dried with sodium sulphate, filtered and evaporated. The crude product was redissolved in dichloromethane (3 ml) and concentrated trifluoroacetic acid (½ ml) was added. The solvent was evaporated and the remaining was purified by preparative HPLC (method A1, Rₜ=9.59 min.) to give the title compound as yellow crystals.

Yield: 77 mg (30%).

HPLC-MS (Method B2): m/z=533 (M+2), Rₜ=3.24 min.

### Example 53

8-(2-(S)-Aminocyclohexyl-(S)-amino)-7-(2-chlorobenzyl)-3-methyl-1-phenethyl-3,7-dihydropurine-2,6-dione. TFA

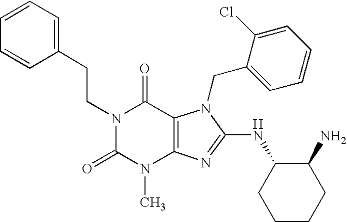

Step A: 8-Bromo-7-(2-chlorobenzyl)-3-methyl-3.7-dihydropurine-2,6-dione. (53A)

8-Bromo-3-methyl-3,7-dihydropurine-2,6-dione (5 g, 20.4 mmol), dimethyl formamide (150 ml), 2-chlorobenzylbromid (2.8 ml, 21.6 mmol), and diisopropylethylamine (7 ml) were reacted and purified as described in example 29, step A, to afford compound 53A as white crystals.

Yield: 6.6 g (88%).

HPLC-MS (Method B2): m/z=371 (M+1), Rₜ=3.031 min.

Step B: 8-Bromo-7-(2-chlorobenzyl)-3-methyl-1-phenethyl-3,7-dihydropurine-2,6-dione (53B)

8-Bromo-7-(2-chlorobenzyl)-3-methyl-3,7-dihydropurine-2,6-dione (53A) (1.5 g, 4.05 mmol), dimethyl formamide (50 ml), 2-bromoethylbenzen (1.48 y, 8.0 mmol), and potassium carbonate (1.68 g, 12.15 mmol) were stirred at 50° C. for 20 hours. The mixture was poured into water (250 ml) and ethyl acetate (20 ml). The precipitate was collected by filtration to afford compound 53B as white crystals.

Yield: 1,43 g (76%).

HPLC-MS (Method B2): m/z=475 (M+2), Rₜ=4.98 min.

Step C: 8-(2-(S)-Aminocyclohexyl-(S)-amino)-7-(2-chlorobenzyl)-3-methyl-1-phenethyl-3,7-dihydropurine-2,6-dione. TFA (53)

8-Bromo-7-(2-chlorobenzyl)-3-methyl-1-phenethyl-3,7-dihydropurine-2,6-dione (53B) (250 mg, 0.528 mmol) and (1S,2S)-(+)-1,2-diamino-cyclohexan (301 mg, 2.64 mmol) were reacted and purified as described in example 52, step C, to give the title compound as white crystals.

Yield: 38 mg (12%).

Prep. HPLC (method A1) Rₜ=9.53 min.

HPLC-MS (Method B2): m/z=507 (M+), Rt=3.32 min.

### Example 54

2-[8-(2-(S)-Aminocyclohexyl-(S)-amino)-7-(2-chlorobenzyl)-3-methyl-2,6-dioxo-1,2,3,6-tetrahydropurin-1-ylmethyl] benzonitrile. TFA

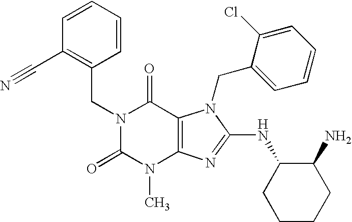

Step A: 2-(8-Bromo-7-(2-chlorobenzyl)-3-methyl-2,6-dioxo-1,2,3,6-tetrahydropurin-1-ylmethyl)benzonitrile (54A)

8-Bromo-7-(2-chlorobenzyl)-3-methyl-3,7-dihydropurine-2,6-dione (53A) (1.5 g, 4.05 mmol), dimethyl formamide (50 ml), alpha bromo-O-tolunitrile (1.59 g, 8.11 mmol), and potassium carbonate (1.68 g, 12.15 mmol) were stirred at 50° C. for 20 hours. The mixture was poured into water (250 ml) and ethyl acetate (20 ml). The precipitate was collected by filtration to afford compound 54A as white crystals.

Yield: 1,66 g (85%).

HPLC-MS (Method B2): m/z=486 (M+2), Rt=4.428 min.

Step B: 2-[8-(2-(S)-Aminocyclohexyl-(S)-amino)-7-(2-chlorobenzyl)-3-methyl-2,6-dioxo-1,2,3,6-tetrahydropurin-1-ylmethyl]benzonitrile. TFA (54)

2-(8-Bromo-7-(2-chlorobenzyl)-3-methyl-2,6-dioxo-1,2,3,6-tetrahydropurin-1-ylmethyl)benzonitrile (54A) (250 mg, 0.516 mmol) and (1S,2S)-(+)-1,2-diamino-cyclohexan (294 mg, 2.58 mmol) were reacted and purified as described in example 52, step C, to give the title compound as yellow crystals.

Yield: 97 mg (30%).

HPLC-MS (Method B2): m/z=518 (M+), Rt=3.105 min.

What is claimed is:

1. A compound of formula II 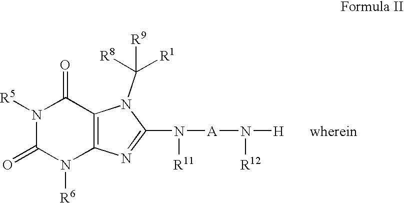
A is C₂-C₆ alkylene; C₂-C₁₀ alkenylene; C₃-C₇ cycloalkylene; C₃-C₇ cycloheteroalkylene; arylene; heteroarylene; C₁-C₂ alkylene-arylene; arylene-C₁-C₂ alkylene; or C₁-C₂ alkylene-arylene-C₁-C₂ alkylene, wherein each alkylene, alkenylene, cycloalkylene, cycloheteroalkylene, arylene, or heteroarylene is optionally substituted with one or more R³ independently;
R¹ is aryl optionally substituted with one or more R² independently, or heteroaryl optionally substituted with one or more R² independently;
R² is H; C₁-C₇ alkyl; C₂-C₇ alkenyl; C₂-C₇ alkynyl; C₃-C₇ cycloalkyl; C₃-C₇ cycloheteroalkyl; —NHCOR³; —NHSO₂R³; —SR³; —SOR³; —SO₂R³; —OCOR³; —CO₂R⁴; —CON(R⁴)₂; —CSN(R⁴)₂; —NHCON(R⁴)₂; —NHCSN(R⁴)₂; —NHCONNH₂; —SO₂N(R⁴)₂; —OR⁴; cyano; —CF₃; nitro; or halogen, wherein each alkyl, alkenyl, alkynyl, cycloalkyl and cycloheteroalkyl is optionally substituted with one or more R³ independently;
R³ is C₁-C₁₀ alkyl; C₂-C₁₀ alkenyl; C₂-C₁₀ alkynyl; C₃-C₇ cycloalkyl; aryl; heteroaryl; OR¹⁰; N(R¹⁰)₂; or SR¹⁰, wherein each alkyl, alkenyl, alkynyl, cycloalkyl, aryl and heteroaryl is optionally substituted with one or more R¹⁰ independently;
R⁴ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; aryl-C₁-C₅ alkylene; heteroaryl; heteroaryl-C₁-C₅ alkylene, —CF₃ or —CHF₂, wherein each alkyl, alkenyl, alkynyl, cycloalkyl, cycloheteroalkyl, aryl, aryl-C₁-C₅ alkylene, heteroaryl, and heteroaryl-C₁-C₅ alkylene is optionally substituted with one or more R¹⁰ independently;
R⁵ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; —OR⁷; aryl-C₁-C₅ alkylene; heteroaryl-C₁-C₅ alkylene; —C₁-C₅-alkyl-C(═O)-aryl; —C₁-C₅-alkyl-C(═O)-heteroaryl; or —[(CH₂)₀-O]ₚ—C₁-C₅ alkyl, wherein o and p are 1-3 independently, and wherein each alkyl, alkenyl, alkynyl, cycloalkyl, cycloheteroalkyl, aryl, heteroaryl, aryl-C₁-C₅ alkylene, —C₁-C₅-alkyl-C(═O)-aryl, —C₁-C₅-alkyl-C(═O)-heteroaryl and heteroaryl-C₁-C₅ alkylene is optionally substituted with one or more R⁷ independently;
R⁶ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; aryl-C₁-C₅ alkylene; heteroaryl-C₁-C₅ alkylene; or C₃-C₇ cycloheteroalkyl-C₁-C₅ alkylene, wherein each alkyl, alkenyl, alkynyl, cycloalkyl, cycloheteroalkyl, C₃-C₇ cycloheteroalkyl-C₁-C₅ alkylene, aryl, heteroaryl, aryl-C₁-C₅ alkylene, and heteroaryl-C₁-C₅ alkylene is optionally substituted with one or more R¹⁰ independently;
R⁷ is H; ═O; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; OR¹⁰; N(R¹⁰)₂; SR¹⁰; cyano; hydroxy; halogen; —CF₃; —CCl₃; —OCF₃; or —OCH₃ wherein each alkyl, alkenyl, alkynyl, cycloalkyl, cycloheteroalkyl, aryl, and heteroaryl is optionally substituted with one or more R¹⁰ independently;
R⁸ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; OR¹⁰; N(R¹⁰)₂; or SR¹⁰, wherein each alkyl, alkenyl, alkynyl, cycloalkyl, cycloheteroalkyl, aryl, and heteroaryl is optionally substituted with one or more R¹⁰ independently;
R⁹ is H; C₁-C₁₀ alkyl optionally substituted with one or more R⁸ independently; or halogen;
R¹⁰ is H; —CF₃; —CCl₃; —OCF₃; —OCH₃; cyano; halogen; —OH, —COCH₃; —CONH₂; —CONHCH₃; —CON(CH₃)₂; —NO₂; —SO₂NH₂; or —SO₂N(CH₃)₂;
if two R⁴ or two R¹⁰ are attached to the same nitrogen they may be connected to form a 3- to 7-membered ring;
R¹¹ is H or C₁-C₆ alkyl optionally substituted with one or more R³ independently;
R¹² is H; C₁-C₆ alkyl optionally substituted with one or more R³ independently; or
If A is C₃-C₇ cycloalkylene or C₃-C₇ cycloheteroalkylene R¹² may be a valence bond between the nitrogen to which R¹² is attached and one of the atoms in the cycloalkylene or cycloheteroalkylene;
or a salt thereof with a pharmaceutically acceptable acid or base.

2. A compound according to claim 1, wherein A is C₂-C₆ alkylene; C₂-C₁₀ alkenylene; C₃-C₇ cycloalkylene; C₃-C₇ cycloheteroalkylene; or arylene, wherein each alkylene, alkenylene, cycloalkylene, cycloheteroalkylene, or arylene is optionally substituted with one or more R³ independently.

3. A compound according to claim 2, wherein A is C₂-C₆ alkylene; C₂-C₁₀ alkenylene; C₃-C₇ cycloalkylene; C₃-C₇ cycloheteroalkylene; arylene; heteroarylene; C₁-C₂ alkylene-arylene; arylene-C₁-C₂ alkylene; or C₁-C₂ alkylene-arylene-C₁-C₂ alkylene, wherein each alkylene, alkenylene, cycloalkylene, cycloheteroalkylene, arylene, or heteroarylene is optionally substituted with one or more R³ independently;
R¹ is aryl optionally substituted with one or more R² independently or heteroaryl optionally substituted with one or more R² independently;
R² is H; C₁-C₇ alkyl; C₂-C₇ alkenyl; C₂-C₇ alkynyl; C₃-C₇ cycloalkyl; C₃-C₇ cycloheteroalkyl; —NHCOR³; —NHSO₂R³; —SR³; —SOR³; —SO₂R³; —OCOR³; —CO₂R⁴; —CON(R⁴)₂; —CSN(R⁴)₂; —NHCON(R⁴)₂; —NHCSN(R⁴)₂; —NHCONNH₂; —SO₂N(R⁴)₂; —OR⁴; cyano; nitro; or halogen, wherein each alkyl, alkenyl, alkynyl, cycloalkyl and cycloheteroalkyl is optionally substituted with one or more R³ independently;
R³ is C₁-C₁₀ alkyl; C₂-C₁₀ alkenyl; C₂-C₁₀ alkynyl; C₃-C₇ cycloalkyl; aryl; heteroaryl; OR¹⁰; N(R¹⁰)₂; or SR¹⁰, wherein each alkyl, alkenyl, alkynyl, cycloalkyl, aryl and heteroaryl is optionally substituted with one or more R¹⁰ independently;
R⁴ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; aryl-C₁-C₅ alkylene; heteroaryl; or heteroaryl-C₁-C₅ alkylene, wherein each alkyl, alkenyl, alkynyl, cycloalkyl, cycloheteroalkyl, aryl, aryl-C₁-C₅ alkylene, heteroaryl, and heteroaryl-C₁-C₅ alkylene is optionally substituted with one or more R¹⁰ independently;
R⁵ is H; C₁-C₁₀alkyl; C₂-C₁₀alkenyl; C₂-C₁₀ alkynyl; C₃-C₇ cycloalkyl; C₃-C₇ cycloheteroalkyl; aryl; heteroaryl; —OR⁷; or —[(CH₂)ₒ—O]ₚ—C₁-C₅ alkyl, wherein o and p are 1-3 independently, and wherein each alkyl, alkenyl, alkynyl, cycloalkyl, cycloheteroalkyl, aryl, and heteroaryl is optionally substituted with one or more R⁷ independently;
R⁶ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl; aryl-C₁-C₅ alkylene; heteroaryl-C₁-C₅ alkylene; or C₃-C₇ cycloheteroalkyl-C₁-C₅ alkylene, wherein each alkyl, alkenyl, alkynyl, cycloalkyl, cycloheteroalkyl, C₃-C₇ cycloheteroalkyl-C₁-C₅ alkylene, aryl, aryl-C₁-C₅ alkylene, heteroaryl, aryl-C₁-Cs alkylene, and heteroaryl-C₁-C₅ alkylene is optionally substituted with one or more R¹⁰ independently;
R⁷ is H; ═O; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; SR¹⁰; cyano; hydroxy; halogen; —CF₃; —CCl₃; —OCF₃; or —OCH₃ wherein each alkyl, alkenyl, alkynyl, cycloalkyl, cycloheteroalkyl, aryl, and heteroaryl is optionally substituted with one or more R¹⁰ independently;
R⁸ is H; C₁-C₁₀ alkyl; C₂-C₁₀ alkenyl; C₂-C₁₀ alkynyl; C₃-C₇ cycloalkyl; C₃-C₇ cycloheteroalkyl; aryl; heteroaryl, OR¹⁰; N(R¹⁰)₂; or SR¹⁰, wherein each alkyl, alkenyl, alkynyl, cycloalkyl, cycloheteroalkyl, aryl, and heteroaryl is optionally substituted with one or more R¹⁰ independently;
R⁹ is H; C₁-C₁₀ alkyl optionally substituted with one or more R⁸ independently; or halogen;
R¹⁰ is H; —CF₃; —CCl₃; —OCF₃; —OCH₃; cyano; halogen; —OH, —COCH₃; —CONH₂; —CONHCH₃; —CON(CH₃)₂; —NO₂; —SO₂NH₂; or —SO₂N(CH₃)₂;
if two R⁴ or two R¹⁰ are attached to the same nitrogen they may be connected to form a 3- to 7-membered ring;
R¹¹ is H or C₁-C₆ alkyl optionally substituted with one or more R³ independently;
R¹² is H; C₁-C₆ alkyl optionally substituted with one or more R³ independently; or
If A is C₃-C₇ cycloalkylene or C₃-C₇ cycloheteroalkylene R¹² may be a valence bond between the nitrogen to which R¹² is attached and one of the atoms in the cycloalkylene or cycloheteroalkylene;
or a salt thereof with a pharmaceutically acceptable acid or base.

4. A compound according to claim 2, wherein A is C₃-C₇ cycloalkylene optionally substituted with one or more R³ independently.

5. A compound according to claim 4, wherein A is cyclohexylene or cycloheptylene, each optionally substituted with one or more R³ independently.

6. A compound according to claim 5, wherein A is cyclohexylene optionally substituted with one or more R³ independently.

7. A compound according to claim 5, wherein A is cyclohexylene or cycloheptylene.

8. A compound according to claim 7, wherein A is cyclohexylene.

9. A compound according to claim 7, wherein A is

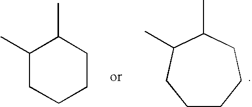

10. A compound according to claim 1, wherein R¹ is aryl optionally substituted with one or more R² independently.

11. A compound according to claim 10, wherein R¹ is phenyl optionally substituted with one or more R² independently.

12. A compound according to claim 1, wherein R₂ is C₁-C₇ alkyl; C₂-C₇ alkynyl; —OR⁴; cyano; —CF₃; or halogen, wherein each alkyl and alkynyl is optionally substituted with one or more R³ independently.

13. A compound according to claim 12, wherein R₂ is C₁-C₇ alkyl; C₂-C₇ alkynyl; cyano; —CF₃; or halogen.

14. A compound according to claim 13, wherein R₂ is cyano, —CF₃ or halogen.

15. A compound according to claim 1, wherein R₂ is C₁-C₇ alkyl; C₂-C₇ alkynyl; cyano; or halogen, wherein each alkyl and alkynyl is optionally substituted with one or more R³ independently.

16. A compound according to claim 15, wherein R₂ is C₁-C₇ alkyl; C₂-C₇ alkynyl; cyano; or halogen.

17. A compound according to claim 16, wherein R₂ is halogen.

18. A compound according to claim 1, wherein R³ is C₁-C₁₀ alkyl or aryl, wherein each alkyl or aryl is substituted with one or more R¹⁰ independently.

19. A compound according to claim 18, wherein R³ is C₁-C₁₀ alkyl or aryl.

20. A compound according to claim 19, wherein R³ is methyl or phenyl.

21. A compound according to claim 1, wherein R⁴ is H; C₁-C₁₀ alkyl, —CHF₂, or aryl, wherein each alkyl or aryl is substituted with one or more R¹⁰ independently.

22. A compound according to claim 21, wherein R⁴ is H; C₁-C₁₀ alkyl, —CHF₂, or aryl.

23. A compound according to claim 22, wherein R⁴ is H, —CHF₂, methyl or phenyl.

24. A compound according to claim 1, wherein R⁴ is H; C₁-C₁₀ alkyl or aryl, wherein each alkyl or aryl is substituted with one or more R¹⁰ independently.

25. A compound according to claim 24, wherein R⁴ is H; C₁-C₁₀ alkyl or aryl.

26. A compound according to claim 25, wherein R⁴ is H, methyl or phenyl.

27. A compound according to claim 1, wherein R⁵ is H; C₁-C₁₀ alkyl; aryl-C₁-C₅ alkylene; —C₁-C₅-alkyl-C(═O)-aryl; or heteroaryl-C₁-C₅ alkylene, wherein each alkyl, aryl-C₁-C₅ alkylene and heteroaryl-C₁-C₅ alkylene is optionally substituted with one or more R⁷ independently.

28. A compound according to claim 27, wherein R⁵ is H; C₁-C₁₀ alkyl optionally substituted with one or more R⁷ independently; —C₁-C₅-alkyl-C(═O)-aryl optionally substituted with one or more R⁷ independently or C₂-C₁₀ alkenyl optionally substituted with one or more R⁷ independently.

29. A compound according to claim 28, wherein R⁵ is H, —C₁-C₅-alkyl-C(═O)-aryl optionally substituted with one or more R⁷ independently or C₁-C₁₀ alkyl optionally substituted with one or more R⁷ independently.

30. A compound according to claim 29, wherein R⁵ is H or —C₁-C₅-alkyl-C(═O)-phenyl optionally substituted with one or more R⁷ independently.

31. A compound according to claim 29, wherein R⁵ is methyl or ethyl optionally substituted with one or more R⁷ independently.

32. A compound according to claim 1, wherein R⁵ is H; C₁-C₁₀ alkyl; aryl-C₁-C₅ alkylene; or heteroaryl-C₁-C₅ alkylene, wherein each alkyl, aryl-C₁-C₅ alkylene and heteroaryl-C₁-C₅ alkylene is optionally substituted with one or more R⁷ independently.

33. A compound according to claim 32, wherein R⁵ is H; C₁-C₁₀ alkyl optionally substituted with one or more R⁷ independently; or C₂-C₁₀ alkenyl optionally substituted with one or more R⁷ independently.

34. A compound according to claim 33, wherein R⁵ is H or C₁-C₁₀alkyl optionally substituted with one or more R⁷ independently.

35. A compound according to claim 34, wherein R⁵ is H.

36. A compound according to claim 31, wherein R⁵ is methyl.

37. A compound according to claim 1, wherein R⁶ is C₁-C₁₀alkyl; aryl-C₁-C₅ alkylene; or heteroaryl-C₁-C₅ alkylene, wherein each alkyl, aryl-C₁-C₅ alkylene and heteroaryl-C₁-C₅ alkylene is optionally substituted with one or more R¹⁰ independently.

38. A compound according to claim 37, wherein R⁶ is C₁-C₁₀alkyl; aryl-C₁-C₅ alkylene; or heteroaryl-C₁-C₅ alkylene.

39. A compound according to claim 37, wherein R⁶ is C₁-C₁₀ alkyl optionally substituted with one or more R¹⁰ independently.

40. A compound according to claim 39, wherein R⁶ is C₁-C₁₀ alkyl.

41. A compound according to claim 39, wherein R⁶ is methyl or ethyl optionally substituted with one or more R¹⁰ independently.

42. A compound according to claim 41, wherein R⁶ is methyl.

43. A compound according to claim 1, wherein R⁷ is H; ═O; C₁-C₁₀ alkyl; C₃-C₇ cycloalkyl; C₃-C₇ cycloheteroalkyl; aryl; heteroaryl, OR¹⁰; N(R¹⁰)₂; SR¹⁰, cyano; or halogen, wherein each alkyl, cycloalkyl, cycloheteroalkyl, aryl, and heteroaryl is optionally substituted with one or more R¹⁰ independently.

44. A compound according to claim 43, wherein R⁷ is ═O; OR¹⁰; C₃-C₇ cycloalkyl; C₃-C₇ cycloheteroalkyl; aryl; heteroaryl; cyano; or halogen, wherein each cycloalkyl, cycloheteroalkyl, aryl, and heteroaryl is optionally substituted with one or more R¹⁰ independently.

45. A compound according to claim 44, wherein R⁷ is ═O; OR¹⁰; cyano; halogen; C₃-C₇ cycloalkyl optionally substituted with one or more R¹⁰ independently or aryl optionally substituted with one or more R¹⁰ independently.

46. A compound according to claim 1, wherein R⁷ is H; ═O; C₁-C₁₀ alkyl; C₃-C₇ cycloalkyl; C₃-C₇ cycloheteroalkyl; aryl; heteroaryl, OR¹⁰; N(R¹⁰)₂; or SR¹⁰, wherein each alkyl, cycloalkyl, cycloheteroalkyl, aryl, and heteroaryl is optionally substituted with one or more R¹⁰ independently.

47. A compound according to claim 46, wherein R⁷ is ═O; C₃-C₇ cycloalkyl; C₃-C₇ cycloheteroalkyl; aryl; or heteroaryl, wherein each cycloalkyl, cycloheteroalkyl, aryl, and heteroaryl is optionally substituted with one or more R¹⁰ independently.

48. A compound according to claim 47, wherein R⁷ is ═O; C₃-C₇ cycloalkyl optionally substituted with one or more R¹⁰ independently or aryl optionally substituted with one or more R¹⁰ independently.

49. A compound according to claim 48, wherein R⁷ is ═O or aryl optionally substituted with one or more R¹⁰ independently.

50. A compound according to claim 49, wherein R⁷ is ═O or phenyl optionally substituted by one or more R¹⁰ independently.

51. A compound according to claim 1, wherein R⁸ is aryl or heteroaryl, wherein each aryl and heteroaryl is optionally substituted with one or more R¹⁰ independently.

52. A compound according to claim 51, wherein R⁸ is aryl or heteroaryl.

53. A compound according to claim 52, wherein R⁸ is phenyl.

54. A compound according to claim 1, wherein R⁹ is H; C₁-C₁₀ alkyl; or halogen.

55. A compound according claim 54, wherein R⁹ is H.

56. A compound according to claim 1, wherein R¹⁰ is H; —CF₃; —OH; cyano; halogen; —OCF₃; or —OCH₃.

57. A compound according to claim 56, wherein R¹⁰ is H; cyano; halogen; or —OCH₃.

58. A compound according to claim 1, wherein R¹¹ is H.

59. A compound according to claim 1, wherein R¹² is H.
